(19) United States
(12) Patent Application Publication
(10) Pub. No.: US 2024/0407372 A1
(43) Pub. Date: Dec. 12, 2024

(54) GRASS ENDOPHYTES

(71) Applicant: RAGT 2N, Rodez (FR)

(72) Inventors: Laure SAINT-PIERRE, Rodez (FR); Marie-Christine GRAS, Rodez (FR)

(21) Appl. No.: 18/745,740

(22) Filed: Jun. 17, 2024

Related U.S. Application Data
(63) Continuation of application No. 17/294,044, filed on May 14, 2021, now Pat. No. 12,048,308.

(30) Foreign Application Priority Data
Nov. 15, 2018 (NZ) 748421

(51) Int. Cl. A01N 63/30; A01H 15/00; C12N 1/14

(52) U.S. Cl. CPC A01N 63/30; A01H 15/00; C12N 1/145

(57) ABSTRACT

A method of improving viability, persistence, and/or vigor of a host plant in the form of Lolium multiflorum Lolium x hybridum and Lolium perenne (host grass) includes artificially inoculating the host grass with an LpTG-1 endophyte RGT15 strain. The LpTG-1 endophyte RGT15 strain can produce at least one alkaloid or combination of alkaloids that provide the host grass with a favorable alkaloid profile and/or protect the plant from biotic stresses.

### PRIORITY AND CROSS REFERENCE TO RELATED APPLICATIONS

This application is a Continuation Application of U.S. application Ser. No. 17/294,044, filed May 14, 2021, which is the U.S. National Stage Application under 35 U.S.C. § 371 of International Application No. PCT/EP2019/081536, filed Nov. 15, 2019, which claims the benefit of New Zealand Patent Application No. 748421, filed Nov. 15, 2018.

### SEQUENCE LISTING IN ELECTRONIC FORMAT

The present application is being filed along with an Electronic Sequence Listing as an XML file in ST.26 format via Patent Center. The Electronic Sequence Listing is provided as a file entitled JAM179.001C1.xml, created and last saved on Jun. 17, 2024, which is 1,439,957 bytes in size, which is replaced by a Replacement Electronic Sequence Listing submitted herewith as a file entitled JAM179.001C1REPLACEMENTSEQLIST.xml, which is 1,441,591 bytes in size and was created on Aug. 27, 2024. The information in the Electronic Sequence Listing is incorporated herein by reference in its entirety.

### TECHNICAL FIELD

The present invention relates to grass endophytes. In particular the present invention relates to fungal endophytes and combinations thereof and uses therefor. More particularly the present invention relates to endophytes which form symbiotic combinations with Lolium multiflorum, Lolium x hybridum and Lolium perenne, ryegrasses. The strains from the present invention are newly discovered distinct strains of Epichloëfestucae var. lolii.

### BACKGROUND ART

Over the last couple of decades, a number of fungal endophytes have been identified in the art as being useful for conferring agronomically useful benefits to artificially infected host grasses [1]. Non-limiting examples include:
AR1, NEA2, NEA6, Edge or AR37.

Prior to the identification of specific fungal endophytes all pasture seed was sold and used with a “standard endophyte” or otherwise called “wild endophyte”. This wild endophyte had an alkaloid profile which contained lolitrem, peramine and ergovaline. This alkaloid profile was known to confer insect resistance to Lolium perenne ryegrass but did unfortunately cause significant toxic effects in animals namely ryegrass staggers and/or heat stress [2, 3]. Hence, the focus on identifying specific endophytes which could confer ongoing specific insect resistance [4, 5, 6] and yet not cause or at least limit the toxic effects for animals.

The initial specific endophyte developed and released commercially by Agresearch was AR1 (an LpTG-1 strain in the taxonomic group Epichloëfestucae var. lolii species [7]) which does not contain lolitrem (the toxin which causes ryegrass staggers) yet via the alkaloid peramine does confer resistance to one key insect Argentine Stem weevil.

Following on from this was the release of NEA type endophytes (other types of LpTG-1 strains [8]) which also don't contain the alkaloid lolitrem but do contain levels of ergovaline which are at levels that generally don't cause heat stress. The Edge endophyte is similar.

In 2004 a new endophyte called AR37 (another type of LpTG-1 strain [9]) was released which contained a different alkaloid called epoxy janthitrem which conferred resistance to a wider range of insects. However, under certain circumstances and in some animal classes, can cause ryegrass staggers.

The inventors have discovered RGT15 which is a new endophyte classified within the taxonomic group Epichloëfestucae var. lolii species LpTG-1 which produces peramine and ergovaline. However, RGT15 has seasonal alkaloid levels which differ from any previous similar endophytes and the inventors believe these differences will confer different insect resistance levels to the ryegrass plant.

The Inventors have also discovered RGT18 which is another unique endophyte classified within the taxonomic group Epichloëfestucae var. lolii species LpTG-3 which produces indole diterpenes called epoxy janthitrems I-IV which confer a wide insect resistance to ryegrass and have not demonstrated any animal side effects [10, 11].

In addition, to the above both these endophytes RGT15 and RGT18 have been shown to have an ideal symbiotic relationship and existence with the R2n™ proprietary and other ryegrass germplasm enabling them each to develop a long-term association which confers agronomically useful benefits for pastoral agriculture.

However, there still remains a need for new endophyte artificially infected grasses to have an alkaloid profile which does not include lolitrem compounds. Lolitrem B has been shown to cause toxicity in the form of Lolium perenne ryegrass staggers in cattle. sheep, deer and horses [2, 3]. Neither RGT15 or RGT18 contain any lolitrem alkaloid.

It is an object of at least one aspect of the present invention to provide a host-grass:endophyte symbiont with a favorable alkaloid profile at least compared to wild type grasses.

There also remains a need for persistent turf grasses to exhibit similar attributes. Persistent turf grasses can also contain higher levels of ergovaline and janthitrems as mechanisms to limit insect pressure and also to stop animal feeding e.g. birds.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DEFINITIONS

The term ‘viability’ as used herein refers to the ability to survive and/or withstand one or more biotic or abiotic stresses.

The term ‘vigour’ as used herein refers to active well-balanced growth.

The term ‘favorable alkaloid profile’ as used herein refers to the presence of an alkaloid, or mixture of alkaloids, produced by an introduced endophyte within at least one host grass at a level or levels which is/are sufficient to be beneficial to: the host grass(es) and/or animals grazing thereon.

The term ‘exemplified’ as used herein refers to the endophyte having a similar alkaloid profile to RGT15 and RGT18.

The term ‘beneficial to the host grass’ as used herein refers to at least one advantage or commercially useful difference conferred to the host grass by the endophyte(s) of the present invention that:
is not present in the wild type host grass; and
is equivalent, superior or different to that conferred by other commercially available ‘host-grass:endophyte’ symbiont cultivars.

For example, in some preferred embodiments a favorable alkaloid profile may result in:
reduced toxicity to animals which graze on the ‘host-grass:endophyte’ symbiont cultivar; and/or
an increased protection against, and/or ability to withstand, abiotic or biotic stresses at least better than wild type host grass.

Preferably, advantages and/or commercially useful differences which may be beneficial to the host grass may include—but should not be limited to—one or more of the following:
providing increased grass persistence/vigour compared to the wild type host plant and/or other commercially available ‘host-plant:endophyte’ symbiont cultivars; and/or
providing protection to biotic and abiotic stresses compared to the wild type host plant and/or other commercially available ‘host-plant:endophyte’ symbiont cultivars; and/or
reducing toxicity to animals, and/or improved animal production, which graze on endophyte grasses of the present invention;
providing a grass which can thrive in a present or future, ecological environment and/or commercial environment, in a manner which at least provides the public with a useful choice.

The term ‘production zone’ refers to any physically defined space, such as a parcel of land; a floor, or part thereof, of a building (which could be multi-storied); or a vehicle (including a trailer or carriage); in which plants can be grown.

The term ‘isolated endophyte’ as used refers to an endophyte removed from its original source and purified from some, most, or all, of the original additional components associated therewith. For example, if the endophyte removed from seeds or plant as found in nature, an isolated endophyte can be considered as an endophyte isolated away from the seeds or plant from which the endophyte was sourced and therefore existing in a purified form. An ‘isolated endophyte’ can therefore be used to inoculate other plants of interest different to that from that in which the endophyte was originally sourced.

### SUMMARY OF THE INVENTION

According to a first aspect of the present invention there is provided a method of improving the viability, persistence and/or vigour of a host plant in the form of Lolium multiflorum, Lolium x hybridum, Lolium perenne, (host grass) which includes the step of:
artificially inoculating the host grass with an LpTG-3 endophyte RGT18 strain;
   so the LpTG-3 endophyte RGT18 strain can:
produce at least one alkaloid or combination of alkaloids that provide the host grass with a favorable alkaloid profile; and/or
protect the plant from biotic stresses.

According to a second aspect there is provided a method substantially as described above wherein the alkaloid is at least one janthitrem epoxide compound.

According to a third aspect there is provided a method substantially as described above wherein the alkaloid is selected from any one of epoxy janthitrem I-IV; or a combination thereof.

According to a fourth aspect there is provided a method of improving the viability, persistence and/or vigour of a host grass in the form of Lolium perenne, Lolium multiflorum Lolium x hybridum host grass) which includes the step of:
artificially inoculating the host grass with an LpTG-1 RGT15 endophyte strain;
   so the LpTG-1 RGT15 endophyte strain can:
produce at least one alkaloid or combination of alkaloids that provide the plant with a favorable alkaloid profile; and/or
protect the plant from biotic stresses.

According to a fifth aspect there is provided a method substantially as described above wherein the alkaloid is selected from a peramine and an ergovaline or a combination thereof.

According to a sixth aspect there is provided a method of improving the viability, persistence and/or vigour of host grasses in the form of Lolium perenne, Lolium multiflorum, Lolium x hybridum comprising the steps of:
a) inoculating a first host grass with RGT15 and
b) inoculating a second host grass with RGT18;
c) co-growing host grasses inoculated as per steps a) and b) or progeny thereof.

Preferably, the first and second host grasses may be selected from the same variety of ryegrass.

According to a seventh aspect there is provided a production zone which includes growing therein at least one host grass in the form of Lolium perenne, annual, Lolium multiflorum, Lolium x hybridum wherein said host grass(es) have been inoculated as per any one of the methods substantially as described above.

According to an eighth aspect there is provided a production zone substantially as described above wherein there is provided two host grasses a first grass inoculated and the second grass inoculated.

According to a ninth aspect there is provided a production zone substantially as described above wherein the two grasses are randomly interspersed within the production zone

According to a 10^{th} aspect there is provided a production zone substantially as described above wherein the two grasses are grown in discrete regions within the production zone.

According to an 11^{th} aspect there is provided a production zone substantially as described above wherein the two grasses are each grown in a respective half of the production zone. According to a 12^{th} aspect there is provided an isolated endophyte of Epichloëfestucae var. lolii species LpTG-1 or LpTG-3 selected from the group consisting of:
RGT15; or
RGT18;

wherein variations in the morphology and/or alkaloid profile over known LpTG-1 and LpTG-3 strains are as exemplified by RGT15 or RGT18 deposited on May 29, 2018 at National Measurement Institute, located at 1/153 BERTIE STREET, PORT MELBOURNE VICTORIA, AUSTRALIA, 3207, and accorded accession numbers V18/011210 and V18/011211, respectively.

According to a 13^{th} aspect of the present invention there is provided a use of an isolated endophyte of Epichloëfestucae var. lolii species LpTG-1 or LpTG-3 selected from the group consisting of:
RGT15; or
RGT18; or
a combination thereof;

to provide seasonal variation in the alkaloids produced by said endophyte(s) when present in a host grass.

According to a 14^{th} aspect of the present invention there is provided a use of an isolated endophyte of Epichloëfestucae var. lolii. species LpTG-1 or LpTG-3 selected from the group consisting of:
RGT15; or
RGT18; or
a combination thereof;
   to accentuate the benefits of increased insect tolerance relative to known endophyte grasses and/or reducing or limiting the side effects of staggers on grazing animals, when present in a host grass.

According to a 15^{th} aspect of the present invention there is provided an isolated endophyte of Epichloëfestucae var. lolii species LpTG-3 RGT18 having a genome including the sequence of nucleic acids as shown in SEQ ID NOs. 1-817.

### BRIEF DESCRIPTION OF THE DRAWINGS

Further aspects of the present invention will become apparent from the ensuing description which is given by way of example only and with reference to the accompanying drawings in which:

FIG. 1 shows a LC-ESI-FTMS extracted ion chromatogram of epoxy-janthitrem I-IV observed in Lolium perenne ryegrass Bronsyn with AR37 endophyte, collected from 0-20 min in positive ionisation mode (ESI+);

FIG. 2A shows a Peramine production (parts per million, ppm) measured in spring, summer and winter for STELLAR-RGT15, RGAS1137-RGT15 and commercially available Lolium perenne ryegrass-endophyte associations. Average production is illustrated by ‘X’;

FIG. 2B shows a ergovalin production (parts per million, ppm) measured in spring, summer and winter for STELLAR-RGT15, RGAS1137-RGT15 and commercially available Lolium perenne ryegrass-endophyte associations. Average production is illustrated by ‘X’;

FIG. 3 shows an Epoxy-janthitrem I production measured in spring, summer and winter for STELLAR-RGT18, RGAS1137-RGT18 and commercially available Lolium perenne ryegrass-AR37 associations. Production is measured as a response (area measured under the peak). Average production is illustrated by ‘X’;

FIG. 4 shows an Epoxy-janthitrem I-IV production measured in summer for STELLAR-RGT18, RGAS1137-RGT18 and commercially available Lolium perenne ryegrass-AR37 associations. Production is measured as a response (area measured under the peak);

FIG. 5 Average epoxy-janthitrem I-IV production measured in summer for STELLAR-RGT18, RGAS1137-RGT18 and Lolium perenne ryegrass-AR37 associations. Production is measured as a response (area measured under the peak);

FIG. 6 shows the differences in the nucleotide sequences of the genotype of RGT18 compared to AR37

FIG. 7 shows baseline tiller count (mean with SE) at day 0 (DPE0);

FIG. 8 shows the damaged tiller count (mean with SE) at DPE10;

FIG. 9 shows the vigour score at DPE10;

FIG. 10 shows the mean damaged tillers (bars indicate SE) at DPE15;

FIG. 11 shows the mean damaged tillers (bars indicate SE) at DPE21;

FIG. 12 shows the percentage of adult aphids survival rate over 8 days;

FIG. 13 shows the numbers of aphid nymphs born over an eight day period;

FIG. 14 shows the number of aphid nymphs surviving over an eight day period;

FIG. 15 shows the percentage of lambs having positive staggers scores after being fed various different grass endophyte symbionts (treatments);

FIG. 16 shows the mean stagger score (Keogh 1973) of lambs by treatment and date;

FIG. 17 shows the average peramine levels on three different dates;

FIG. 18 shows the average lolitrem B levels on three different dates;

FIG. 19 shows the average epoxy janthitrem I levels on three different dates;

FIG. 20 shows the results of the Lennox Trial assessing: Average Total Dry Matter Yield (kgDM/ha) by Years; and

FIG. 21 shows the results of the Waikeria Trial assessing: Average Total Dry Matter Yield (kgDM/ha) by Years.

### DETAILED DESCRIPTION

### Example 1

### SSR Genotyping (B Loci Methods)

DNA was extracted from each sample using the MagAttract Plant DNA kit (Qiagen, Hilden, Germany). The endophyte-specific SSR markers, B10 and B11 were used for PCR-based in planta detection of endophytes. PCR amplifications were performed in 12.5 μl reaction volumes containing 1 μL genomic DNA (5 ng/μl), 1× PCR buffer (Bioline, London, UK), 0.2 μM of forward and reverse primer (Sigma Aldrich, St. Louis, Missouri, USA; Applied Biosystems, Foster City, California, USA), 0.125 μl of 5 mM dNTP (GE Healthcare, Chalfont St Giles, UK), 1 U immolase (Bioline), and was processed in a thermocycler (DNA Engine, Bio-Rad, Hercules, California, USA) programmed for 10 min at 95° C. followed by 30 cycles of 1 min at 94° C., 2 min at 65° C., 1 min at 72° C., then 10 min at 72° C.

The PCR products were diluted with sterile Milli-Q water (1:10), and 2 μl of the diluted product was mixed with 8 μl of ABI 3730 loading solution [7.95 μl of Hi-Di™ formamide (Applied Biosystems, Foster City, California, USA), 0.05 μl of 500Liz™ (Applied Biosystems, Foster City, California, USA) size standard] before analysis on the ABI 3730xl automated capillary electrophoresis platform (Applied Biosystems Biosystems, Foster City, California, USA). Products were detected using the GeneMapper version 3.7 software (Applied Biosystems Biosystems, Foster City, California, USA), and endophyte samples were identified by the presence of amplification peaks specific for each SSR marker.

TABLE 1
SSR marker profile of selected endophyte
strains at the B10 and B11 loci [7].

| | | | | B11 (bp) | |
|---|---|---|---|---|---|
| Endophyte | Taxonomic | Alkaloid | B10 | Allele | Allele |
| strain | group | profile | (bp) | 1 | 2 |
| RAGT/R2n | | | | | |
| Endophytes | | | | | |
| 1.4.4 | LpTG-1 | EP | 176 | 238 | |
| RGT18 | LpTG-3 | J | 162 | 131 | |
| RGT15 | LpTG-1 | EP | 176 | 152 | 156 |
| 1.9.7 | LpTG-1 | EP | 176 | 160 | 164 |
| 2.5.3 | LpTG-1 | EP | 176 | 152 | 156 |
| 1.11.13 | LpTG-1 | EP | 176 | 172 | |
| Commercial | | | | | |
| Endophytes | | | | | |
| NEA6 | LpTG-1 | EP | 176 | 199 | |
| AR37 | LpTG-3 | J | 162 | 131 | |
| SE | LpTG-1 | LEP | 176 | 176 | |
| AR1 | LpTG-1 | P | 176 | 148 | |

LpTG-Lolium perenne Taxonomic Group;
E-ergovaline;
P-peramine;
J-janthitrems;
L-lolitrem B;
bp-base pair

### Example 2: Genome Sequencing to Identify SNP Variation between RGT18 and AR37

### 2.1 Illumina Platform Sequencing

### DNA Extraction

DNA for genome survey sequencing was extracted from lyophilized mycelia using a cetyltrimethylammonium bromide (CTAB) based extraction method (Möller et al. 1992), and the quality and quantity of the DNA was assessed by both agarose gel electrophoresis and specific absorbance measurements using the NanoDrop 2000 Spectrophotometer (Thermo Scientific).

Prior to sequencing, genetic identities of isolated endophyte strains were confirmed using endophyte specific SSR markers.

### Paired-End Library Preparation and Sequencing

Genomic DNA was fragmented in a Covaris instrument (Woburn, MA, USA) to an average size of 100-900 bp.

For the RGT18 endophyte DNA sample, paired-end libraries with inserts c. 400 bp in size were prepared using the standard protocol (TruSeq DNA Sample Prep V2 Low Throughput: Illumina Inc., San Diego, USA) with paired-end adaptors. Library quantification was performed using the KAPA library quantification kit (KAPA Biosystems, Boston, USA). Paired-end libraries were pooled according to the attached adaptors and sequence analysed using the HiSeq2000 platform (Illumina) following the standard manufacturer's protocol, RGT18, was sequenced in September 2012.

For AR37 endophyte DNA, paired-end libraries with inserts c. 400 bp in size were prepared using the standard protocol (Nextera DNA Library Preparation Kit: Illumina Inc., San Diego, USA) with paired-end adaptors. Library quantification was performed using the KAPA library quantification kit (KAPA Biosystems, Boston, USA). Paired-end libraries were pooled according to the attached adaptors and sequence analysed using the MiSeq platform (Illumina) following the standard manufacturer's protocol. The AR37 isolate was sequenced in September 2012.

All generated sequence reads were quality controlled by filtering and trimming of reads based on quality using a custom Python script, which calculates quality statistics, and stores trimmed reads in several fastq files.

2.2. SNP Variation between AR37 and RGT18

### 2.2.1. AR37 Reference Genome

A reference genome for AR37 was assembled using SOAPdenovo. A total of 9,604, 168 paired-end reads generated using Illumina sequencing technology were assembled to generate a reference genome with 19982 scaffold sequences (Table 2). To minimise ‘N’s present in the assembly ‘command-line resolve’ was used, thus generating the AR37 resolved reference genome.

TABLE 2
Sequencing statistics for the AR37 reference genome

| Sequence statistic | Length (bp) |
|---|---|
| Size_includeN | 31354167 |
| Size_withoutN | 29768040 |
| Scaffold_Num | 19982 |
| Mean_Size | 1569 |
| Median_Size | 115 |
| Longest_Seq | 151941 |
| Shortest_Seq | 100 |
| Singleton_Num | 13980 |
| Average_length_of_break(N)_in_scaffold | 79 |

### 2.2.2 Mapping Reads to the AR37 Reference Genome

For read mapping to reference sequences, raw fastq sequences generated by the Illumina sequencers were trimmed and quality filtered using the Gydle ‘nuclear’ program in filter mode. The settings used trims reads to a minimum length of 50 bp having greater than Phred score equivalent of 20 and removed Illumina adapters. Reads were mapped to the AR37 reference genome using the Gydle ‘nuclear’ program in search mode. The settings used were Minimum length (L) of High Scoring Pairs 50, Sensitivity (s-minimum consecutive identities within a HSP) 25, K-mer size (k) used for search, 13 Mismatches allowed (m) 6, and—random-best (Reports a single hit randomly selected among the best hits).

AR37 and RGT18 reads were mapped to the AR37 reference genome using Gydle to generate a gym file. These were generated from the ‘nuclear’ aligned reads using the Gydle ‘gym-build’ program. SNPs were identified using the Gydle program findsnp which uses the gym file as an input. Settings for findsnp were min-coverage 2 (i.e. at least 2 DNA reads had to cover the base of interest) and a min-allele-frequency of 80% (i.e. a base different to the reference had to be seen in a least 80% of the reads covering that base) and min-allele-count 2 (to count SNPs in contigs with unresolved repeats per strain). SNPs were then further filtered such that: 1) SNPs with a minimum allele frequency >80% were excluded when comparing AR37 (reference) v AR37 (reads); 2) contigs had to be greater than 999 bp; and 3) less than 20 minimum-allele-count 2 AR37 (reference) v AR37 (reads) SNPs allowed per contig.

Using this approach, the inventors identified 817 SNPs, with an average coverage (sequence read depth) of 18×, which differentiate clearly between AR37 and RGT18 (FIG. 7).

### Example 3: Generation of Novel Host-Endophyte Associations

Lolium perenne ryegrass-endophyte associations were generated by inoculation using the ‘cut and stuff’ method [12]. Infected plants were identified 6-8 weeks post inoculation using strain specific DNA-based diagnostics to confirm endophyte presence and identity. A total of 154 novel Lolium perenne ryegrass-endophyte associations were generated (Table 3).

TABLE 3
Variety-endophyte associations generated via
endophyte inoculation into seedling meristem.

| Host | Endophyte | Number of |
|---|---|---|
| variety | strain | genotypes |
| STELLAR | RGT15 | 44 |
| STELLAR | RGT18 | 44 |
| RGAS1137 | RGT15 | 30 |
| RGAS1137 | RGT18 | 36 |
| Total | | 154 |

### Example 4: Vegetative Stability of Endophytes

Vegetative stability of the associations was assessed six monthly, over a 24-month period (Table 4). Three or more tiller samples (c. 0.5 cm from the base) were harvested from each plant after 6, 12, 18 and 24 months growth in the field. DNA was extracted from freeze dried samples using the MagAttract Plant DNA kit (Qiagen, Hilden, Germany). Each sample was analysed for endophyte strain presence and identity using strain specific DNA-based diagnostics.

Table 3 summarises the number of endophyte positive plants identified 6, 12, 18 and 24 months following successful infection determined at 6 weeks post inoculation. Once novel Lolium perenne ryegrass-endophyte associations are established they remain stable. Further, endophyte strain RGT18 exhibited higher vegetative stability compared to strain RGT15. However, this could also have been due to the varietal-endophyte symbiosis occurring between this endophyte and the host variety, but also the inventors also believe could be due to the compatibility/marriage ability of the RGT18 endophyte with a host grass.

TABLE 4
Vegetative stability of variety-endophyte associations. Endophyte
infected plants were identified at 6 weeks post inoculation,
followed by 6-monthly assessments of association stability.

| | | Host variety + Endophyte strain Inoculated | | | | |
|---|---|---|---|---|---|---|
| | | Stellar + | Stellar + | RGAS1137 + | RGAS1137 + | |
| | | RGT15 | RGT18 | RGT15 | RGT18 | Total |
| 6 weeks | Endophyte | 44/47 | 44/44 | 30/30 | 36/36 | 154/157 |
| | detected | | | | | |
| | Vegetative | 94% | 100% | 100% | 100% | |
| | stability | | | | | |
| 6 months | Endophyte | 35/44* | 37/42* | 25/30* | 33/34 | 130/150 |
| | detected | | | | | |
| | Vegetative | 79.5% | 88.1% | 83.3% | 97.1% | |
| | stability | | | | | |
| 12 months | Endophyte | 36/45 | 40/43 | 23/31* | 28/34 | 127/153 |
| | detected | | | | | |
| | Vegetative | 80.0% | 93.0% | 74.2% | 82.4% | |
| | stability | | | | | |
| 18 months | Endophyte | 36/45 | 39/43 | 24/32 | 30/34 | 141/154 |
| | detected | | | | | |
| | Vegetative | 80.0% | 90.7% | 75.0% | 88.2% | |
| | stability | | | | | |
| 24 months | Endophyte | 37/45 | 40/43 | 23/32 | 28/33* | 134/153 |
| | detected | | | | | |
| | Vegetative | 82.2% | 93.0% | 71.9% | 84.8% | |
| | stability | | | | | |

*Where total plants tested is lower than the number of plants in the field, some plants were either not tested or deceased.

### Example 5: Intergenerational Stability

The intergenerational stability of the endophytes was assessed in seed generated via pollen cloud experimentation. Analysis was performed using a bulked seed approach whereby 10 seeds were pooled to form 1 sample. DNA extraction was performed using the DNeasy 96 plant DNA extraction kit (Qiagen, Hilden, Germany) for each seed line. Each sample was analysed for endophyte strain presence and identity using strain specific DNA-based diagnostics.

Intergenerational stability, measured as transmission of endophyte to seed, showed endophyte strain RGT18 exhibits higher stability compared to strain RGT15 (Table 5). Variation for intergenerational stability was also observed between varieties; host variety RGAS1137 transmits endophyte at a higher frequency compared to STELLAR (Table 5).

TABLE 5
Intergenerational stability of variety-endophyte associations,
measured as transmission of endophyte to seed.

| | Endo- | Endo- | Endo- | Total | Inter- |
|---|---|---|---|---|---|
| Host | phyte | phyte | phyte | genotypes | generational |
| variety | strain | detected | negative | tested | stability |
| STELLAR | RGT15 | 40 | 11 | 51 | 78% |
| STELLAR | RGT18 | 39 | 5 | 44 | 89% |
| RGAS1137 | RGT15 | 30 | 6 | 36 | 83% |
| RGAS1137 | RGT18 | 35 | 1 | 36 | 97% |
| Total | | 144 | 23 | 167 | |

### Example 6: Alkaloid Profiling

The alkaloid profiles (ergovaline, peramine and janthitrems) of the associations were assessed over a growing season under field conditions to gain insight into seasonal fluctuations and presence of alkaloids.

### 6.1 Methods

### Sample Harvest

Plant material for alkaloid analysis was harvested approximately 5 cm above ground. Fresh material was packed into paper bags and stored at −80° C. Samples were then freeze dried, ground to a fine powder and stored at 21° C. in the dark.

### Sample Preparation for Alkaloid Analysis

Freeze-dried and ground Lolium perenne ryegrass-endophyte samples harvested from three different seasonal periods were measured for alkaloid content.

Plant material (20 mg±0.2 mg) was extracted twice with 1 mL of methanol:water (80:20, v:v) (Merck LiChrosolv ≥99.9%; MilliQ water). The supernatants were combined and dried using a SpeedVac Concentrator (Thermo Fisher Scientific, Savant SPD 2010) at room temperature for approximately 16 h, and reconstituted in 200 μL of methanol:water (80:20, v:v) containing Ergotamine D-tartrate (Sigma-Aldrich, St Louis, USA) as an internal standard at a concentration of 216 ng/ml.

For alkaloid quantitation, peramine nitrate (BDG Synthesis, Wellington, NZ) and ergovaline were used to construct concentration curves from 1 to 2000 ng/ml (peramine and ergovaline) in matrix (endophyte free Lolium perenne ryegrass plant).

### LCMS Parameters

### HPLC Analysis

Extracts were analysed using a 100 mm×2.1 mm Themo Hypersil Gold 1.9 μm HPLC column fitted to a Thermo Fischer Scientific Vanquish liquid chromatograph (Thermo Fischer Scientific, Bremen). The compounds were detected with a Thermo Fisher QExactive Plus mass spectrometer (Waltham, MA, USA; Thermo, Bremen, Germany), operating in the ESI mode with a HESI probe for positive data acquisition.

### Mass Spectrometry Analysis

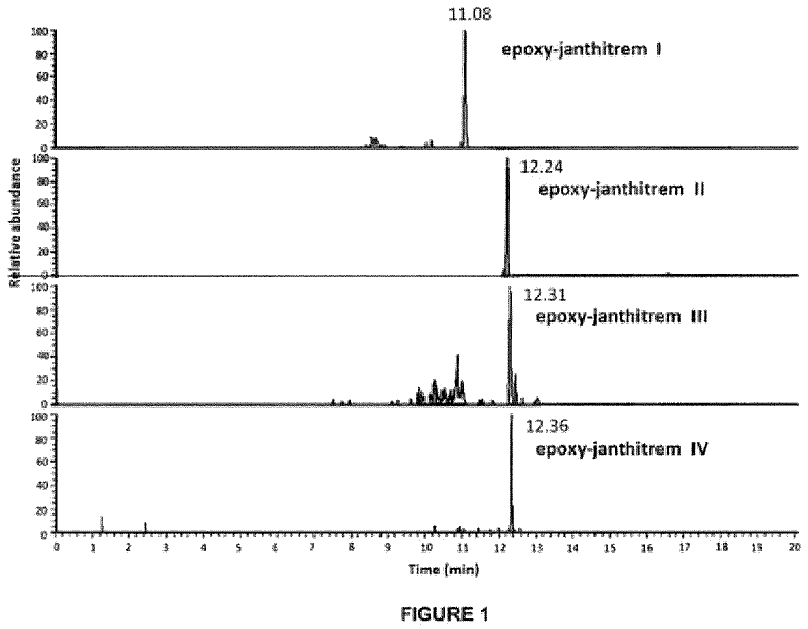

The sample extract (3 μL) was assessed in FT positive mode over a mass range of 80-1200 amu with resolution set at 35,000. Typical mass accuracy for the alkaloids was 3-5 ppm. Relative quantitation (expressed as peak area) were determined for epoxy-janthitrems I to IV (FIG. 1). These were identified based on mass accuracy and fragmentation patterns via LCMS/MS (Table 6).

TABLE 6
Targeted LCMS/MS analysis of epoxy-janthitrem I-IV indicating
accurate masses (m/z), retention times (RT) and MSn fragmentation
data (LC-MS/MS) which were acquired in positive ionisation
mode [M + H] using a Thermo Fisher Q-Exactive
Plus orbitrap mass spectrometer. Accurate mass and MSn fragmentation
results were compared with theoretical masses and fell within
the range of 5 ppm difference (Delta ppm).

| | | Epoxy- | Epoxy- | Epoxy- | Epoxy- |
|---|---|---|---|---|---|
| | | janthitrem | janthitrem | janthitrem | janthitrem |
| | Metabolite | I | II | III | IV |
| | m/z [M + H] | 646.3735 | 670.4076 | 672.4230 | 714.4341 |
| | RT (min) | 11.08 | 12.24 | 12.31 | 12.36 |
| | 1 | 222.1277 | 222.1275 | 222.1274 | 222.1278 |
| Production: | 2 | 280.1696 | 280.1692 | 280.1692 | 280.1694 |
| LC-MS/ | 3 | 588.3320 | 612.3676 | 614.3833 | 656.3934 |
| MS | 4 | 631.3459 | 655.3814 | 657.3969 | 699.4081 |
| | Chemical | C39 H52 | C42 H56 | C42 H58 | C44 H60 |
| | Formula | O7 N | O6 N | O6 N | O7 N |
| | [M + H] | | | | |
| | Theoretical | 646.3738 | 670.4102 | 672.4259 | 714.4364 |
| | Mass | | | | |
| | [M + H] | | | | |
| | Delta (ppm) | −0.5 | −3.8 | −4.3 | −3.3 |

### 6.2 RGT15

STELLAR-RGT15 and RGAS1137-RGT15 were measured for peramine and ergovaline production in spring, summer and winter. Lolitrem B was not observed in any variety-RGT15 associations.

Generally, higher average concentrations of peramine and ergovaline were observed in summer compared to spring and winter (Table 7). STELLAR-RGT15 associations exhibit less seasonal variation in peramine and ergovaline concentrations compared to RGAS1137-RGT15 associations. This higher concentration exhibited in summer is very positive in providing high levels of insect ressitance at a time of the year when insect presence and lifecycles are at their peak in pastoral systems.

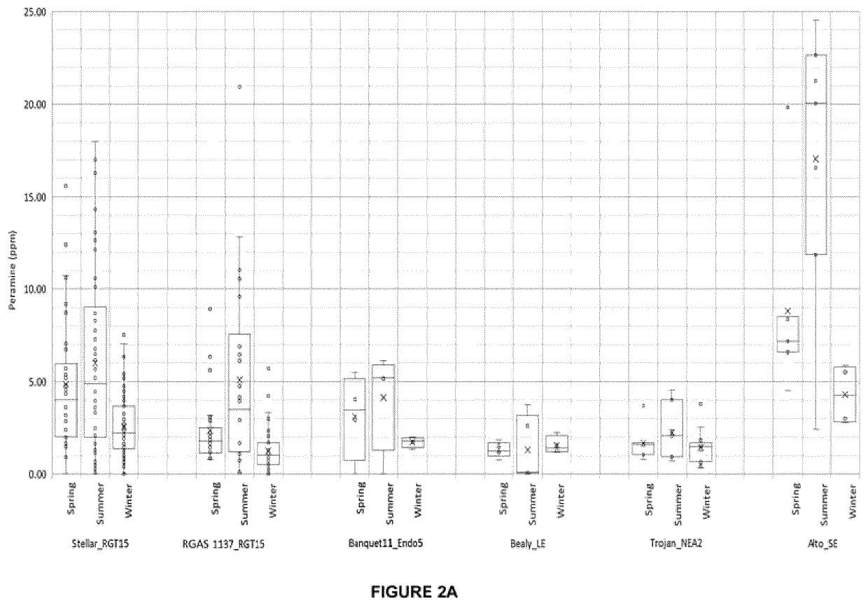

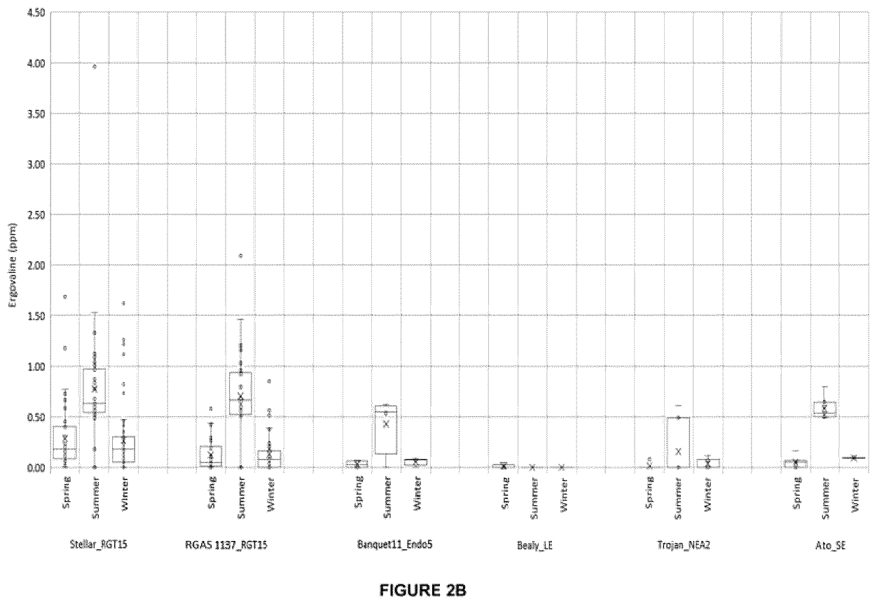

A wide range in alkaloid production was observed within each population (FIG. 2a-b). Individual genotypes within STELLAR and RGAS1137 were identified with peramine concentrations higher than commercial cultivars tested.

TABLE 7
Seasonal averages for ergovaline and peramine production
for Lolium perenne ryegrass-endophyte associations.

| | | Number of | Average | Average |
|---|---|---|---|---|
| | | genotypes | ergovaline | peramine |
| Association | Season | measured | (mg/kg) | (mg/kg) |
| STELLAR-RGT15 | Spring | 42 | 0.28 | 4.85 |
| RGAS1137-RGT15 | Spring | 26 | 0.12 | 2.34 |
| Banquet-Endo5 | Spring | 4 | 0.03 | 3.10 |
| Bealey -LE | Spring | 4 | 0.01 | 1.30 |
| TROJAN-NEA2 | Spring | 7 | 0.01 | 1.66 |
| Alto-SE | Spring | 7 | 0.03 | 8.79 |
| STELLAR-RGT15 | Summer | 42 | 0.77 | 5.98 |
| RGAS1137-RGT15 | Summer | 26 | 0.70 | 5.07 |
| Banquet-Endo5 | Summer | 4 | 0.43 | 5.07 |
| Bealey -LE | Summer | 4 | 0.00 | 1.30 |
| TROJAN-NEA2 | Summer | 7 | 0.16 | 2.23 |
| Alto-SE | Summer | 7 | 0.58 | 17.04 |
| STELLAR-RGT15 | Winter | 36 | 0.27 | 2.51 |
| RGAS1137-RGT15 | Winter | 25 | 0.13 | 1.26 |
| Banquet-Endo5 | Winter | 4 | 0.07 | 1.72 |
| Bealey -LE | Winter | 4 | 0.00 | 1.54 |
| TROJAN-NEA2 | Winter | 14 | 0.04 | 1.41 |
| Alto-SE | Winter | 4 | 0.09 | 4.27 |

To note:
the ergovaline levels exhibited in RGT15 were at optimum levels for suppressing insects but not at a level to cause animal effects.

### 6.3 RGT18

STELLAR-RGT18 and RGAS1137-RGT18 were measured for epoxy-janthitrems I-IV production in spring, summer and winter (Table 8).

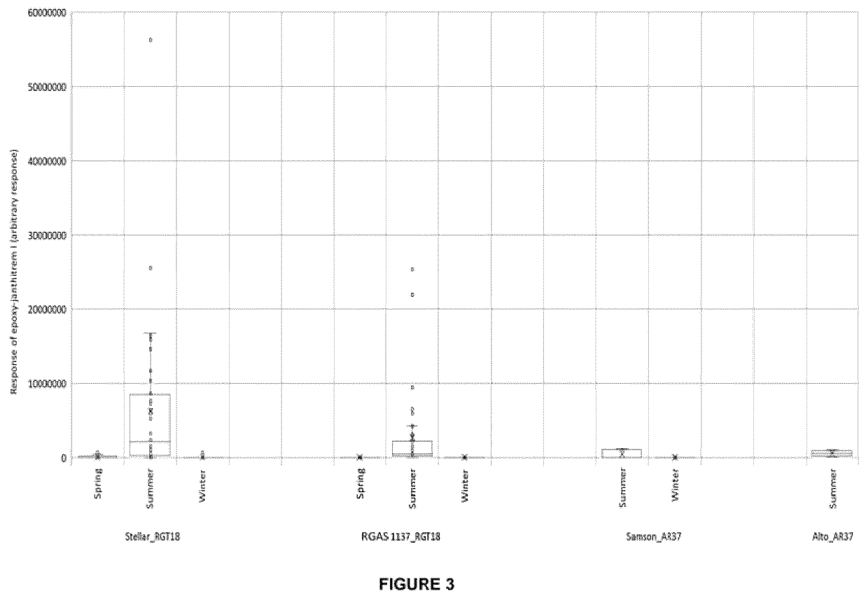

A wide range in alkaloid production was observed within each population (FIG. 3). Individual genotypes within STELLAR and RGAS1137 were identified with epoxy-janthitrem I production concentrations higher than commercial cultivars tested, as were genotypes with epoxy-janthitrem concentrations lower than commercial cultivars tested.

Lower average concentrations of epoxy-janthitrem I were observed in spring and winter compared to summer. Again this is positive, in that in pastoral systems have higher insect pressures exhibited in summer, than in cooler times of the year.

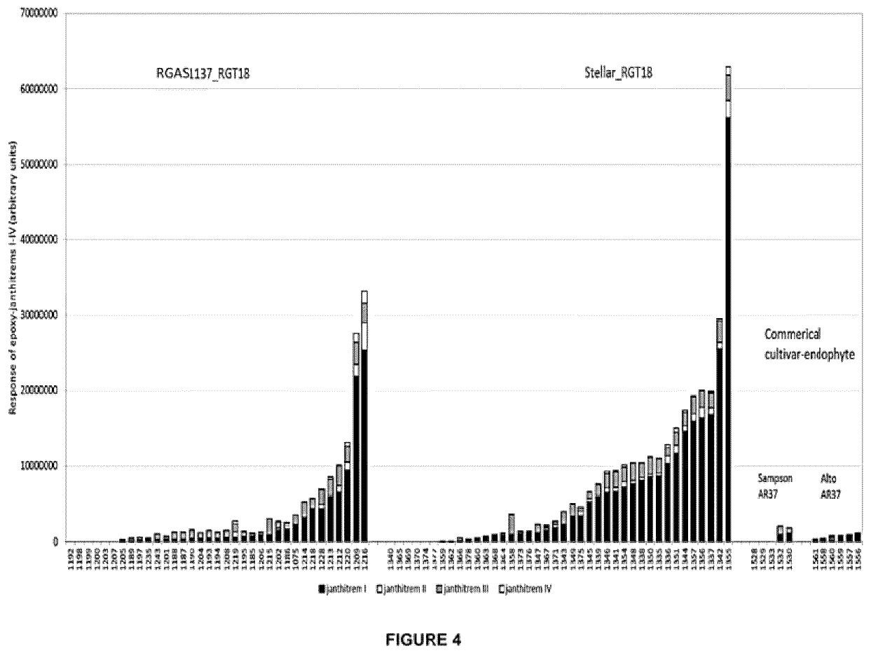

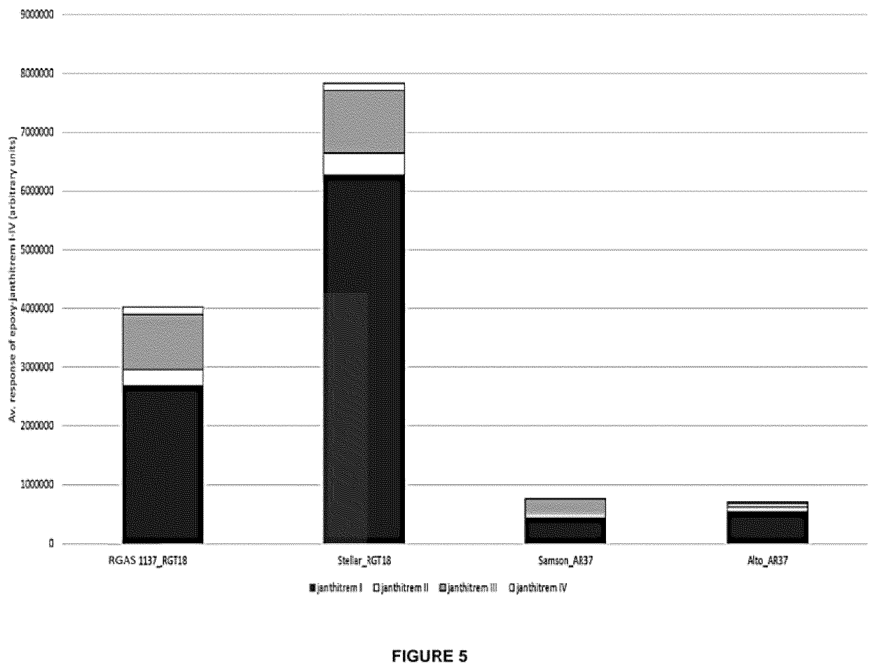

The production of epoxy-janthitrem I and its variants (epoxy-janthitrems II-IV) in the summer were also measured (FIG. 4). Average levels were greater in STELLAR-RGT18 compared to RGAS1137-RGT18 (FIG. 5). Epoxy-janthitrem I was dominant in all samples tested, followed by epoxy-janthitrem III (Table 9).

TABLE 8
Seasonal averages for janthitrem I production for
Lolium perenne ryegrass-endophyte associations

| | | Number of | Average |
|---|---|---|---|
| | | genotypes | janthitrem I |
| Association | Season | measured | (arbitrary units) |
| STELLAR-RGT18 | Spring | 43 | 100116 |
| RGAS1137-RGT18 | Spring | 38 | 22054 |
| SAMSON-AR37 | Spring | 5 | 0 |
| STELLAR RGT18 | Summer | 43 | 6268565 |
| RGAS1137-RGT18 | Summer | 38 | 2677393 |
| SAMSON-AR37 | Summer | 5 | 434659 |
| Alto-AR37 | Summer | 6 | 538814 |
| STELLAR RGT18 | Winter | 36 | 42794 |
| RGAS1137-RGT18 | Winter | 32 | 4473 |
| SAMSON-AR37 | Winter | 8 | 0 |

TABLE 9
The proportion of epoxy-janthitrems I-IV (%) measured in Lolium perenne
ryegrass-endophyte associations during the summer season.
Proportion of epoxy-janthitrems I-IV (%)

| | | epoxy- | epoxy- | epoxy- | epoxy- |
|---|---|---|---|---|---|
| | | janthitrem | janthitrem | janthitrem | janthitrem |
| Association | Season | I | II | III | IV |
| RGAS1137- | Summer | 66.61 | 6.91 | 23.38 | 3.10 |
| RGT18 | | | | | |
| STELLAR- | Summer | 79.96 | 4.78 | 13.65 | 1.61 |
| RGT18 | | | | | |
| SAMSON- | Summer | 57.08 | 0.00 | 42.92 | 0.00 |
| AR37 | | | | | |
| Alto-AR37 | Summer | 76.30 | 10.36 | 9.89 | 3.45 |

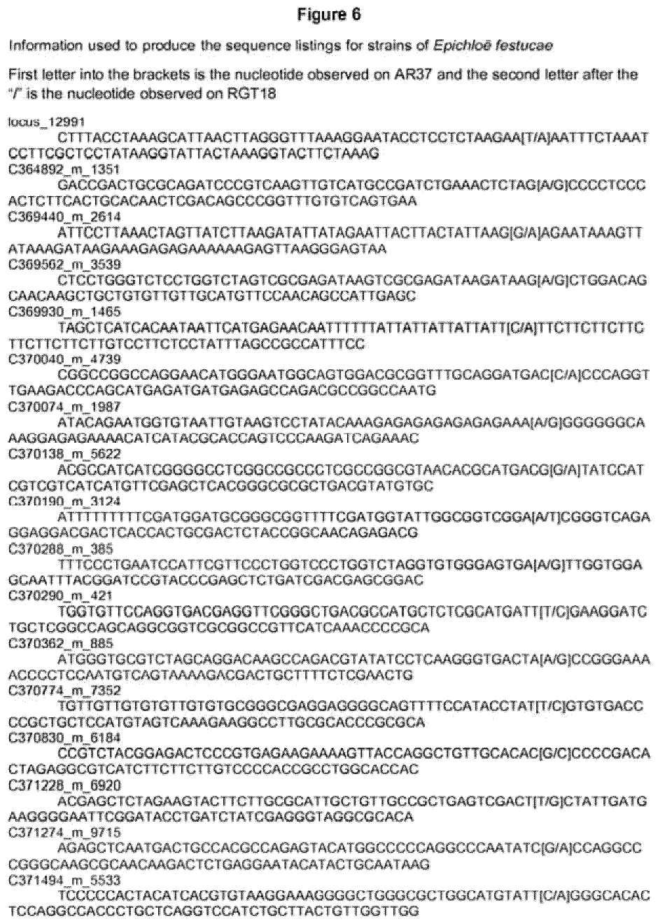

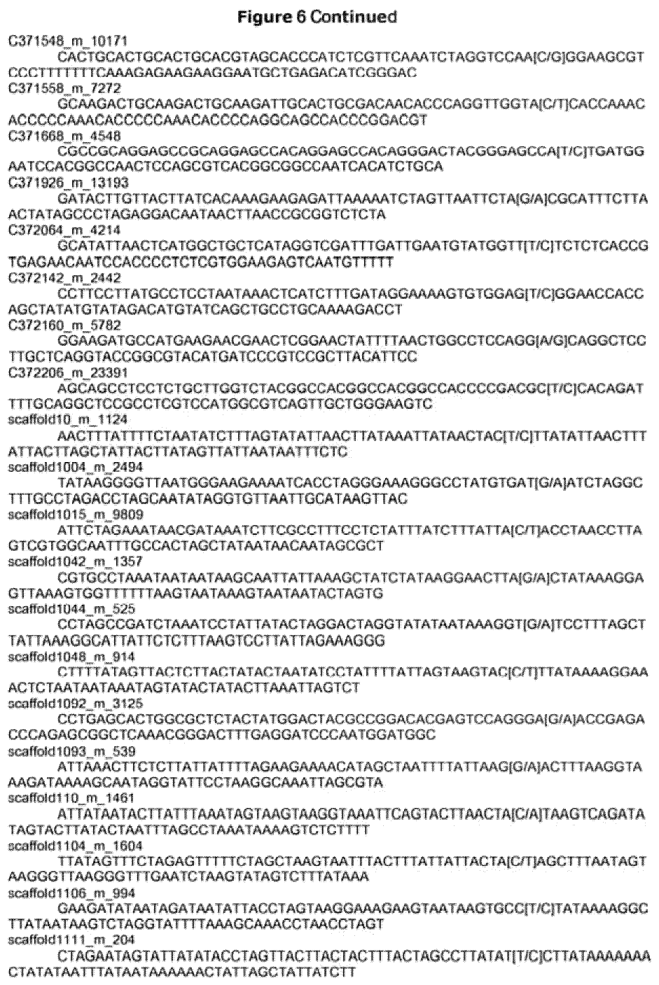

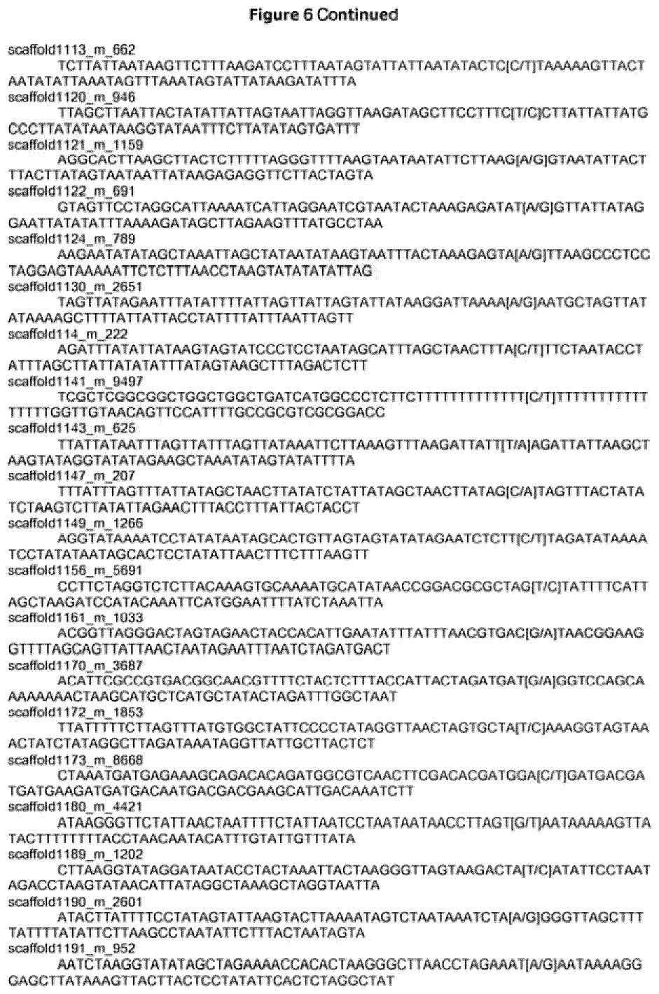

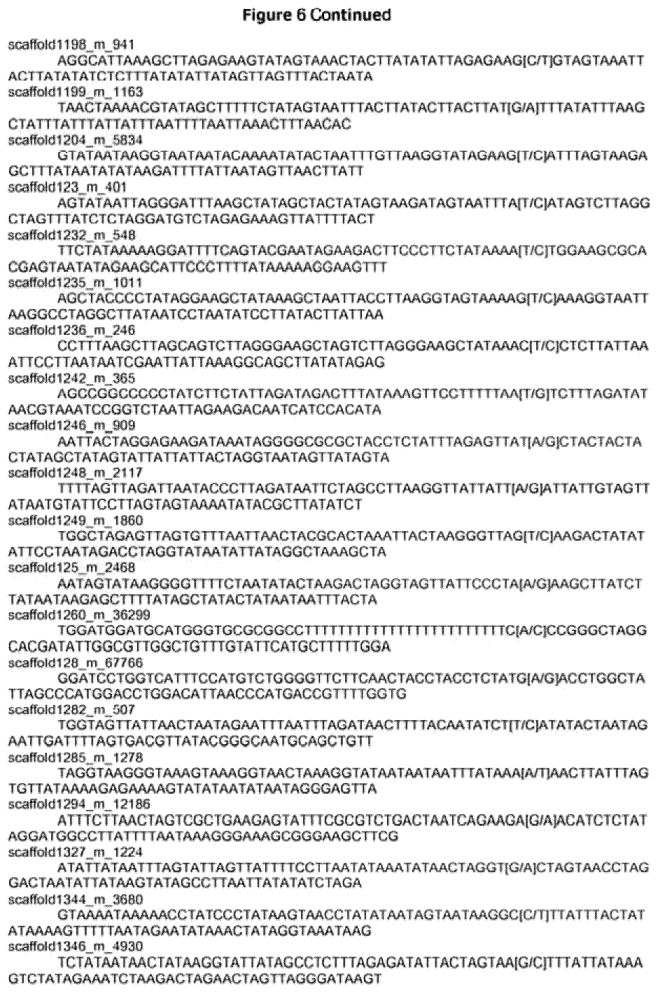

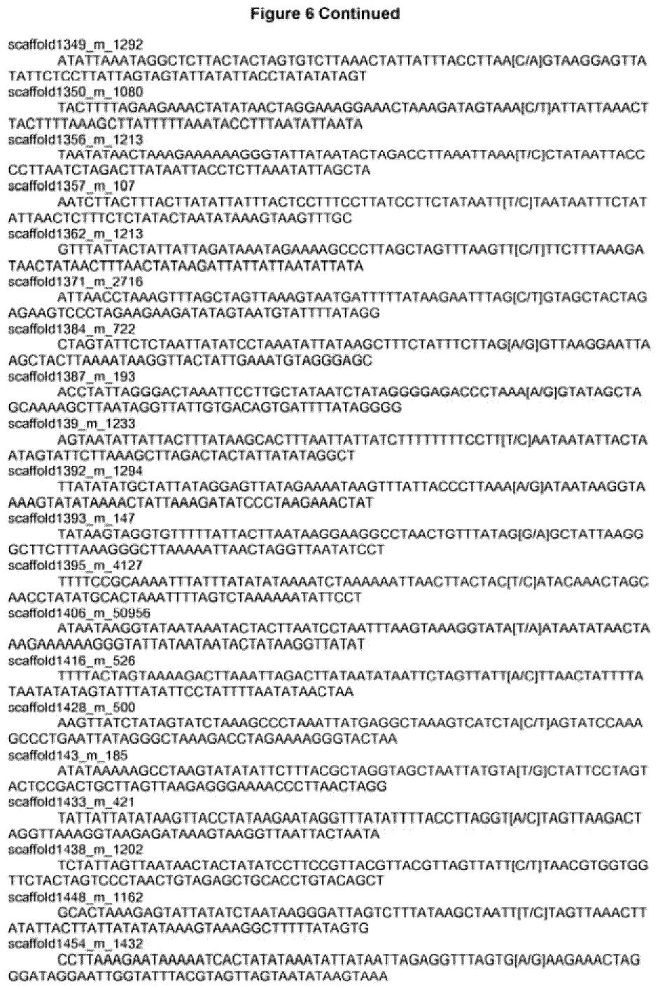

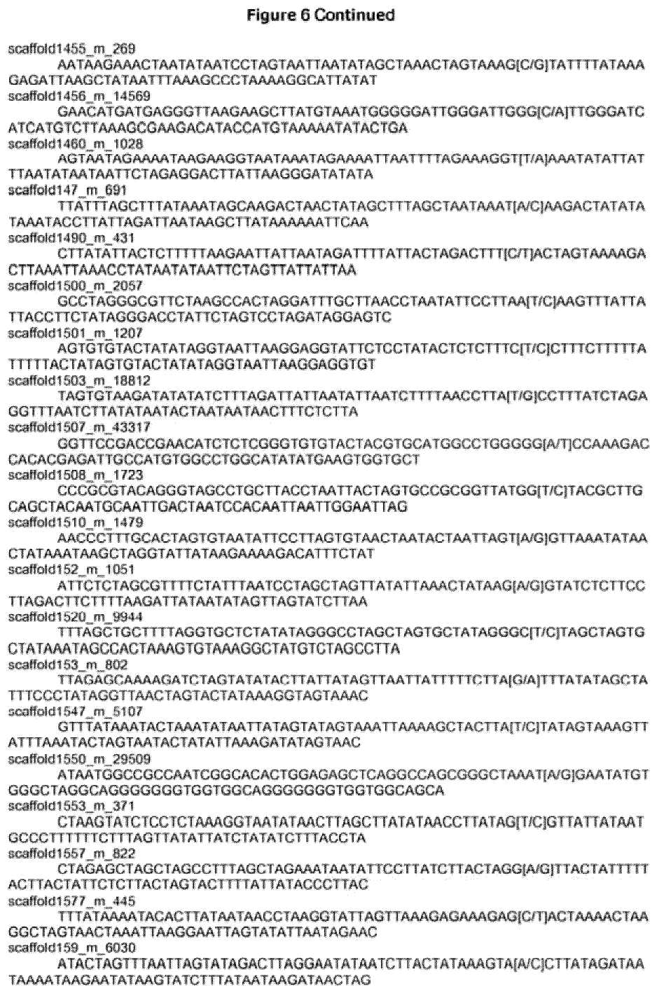

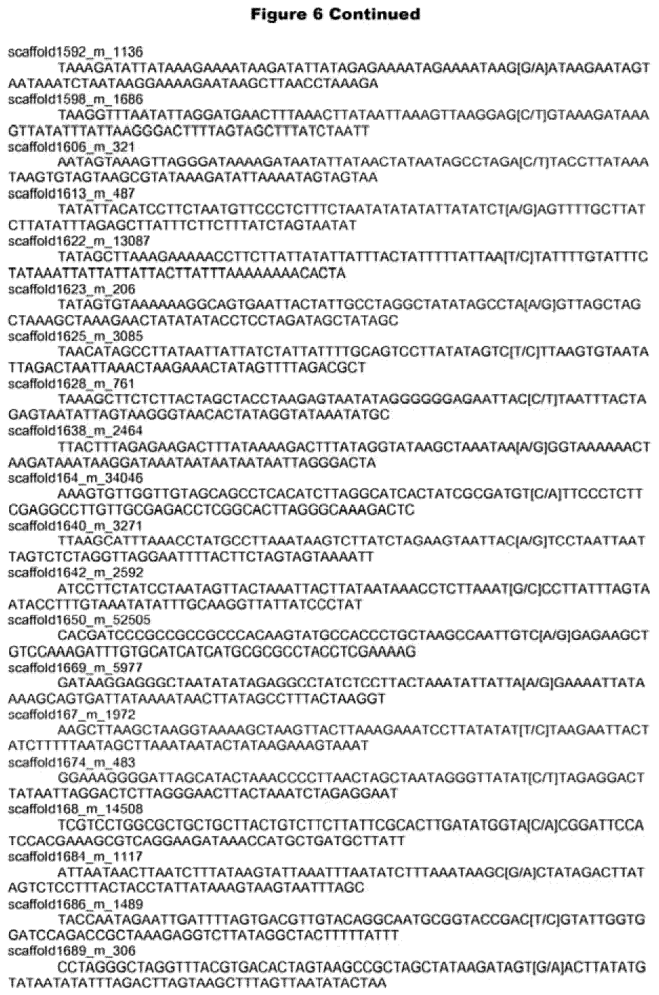

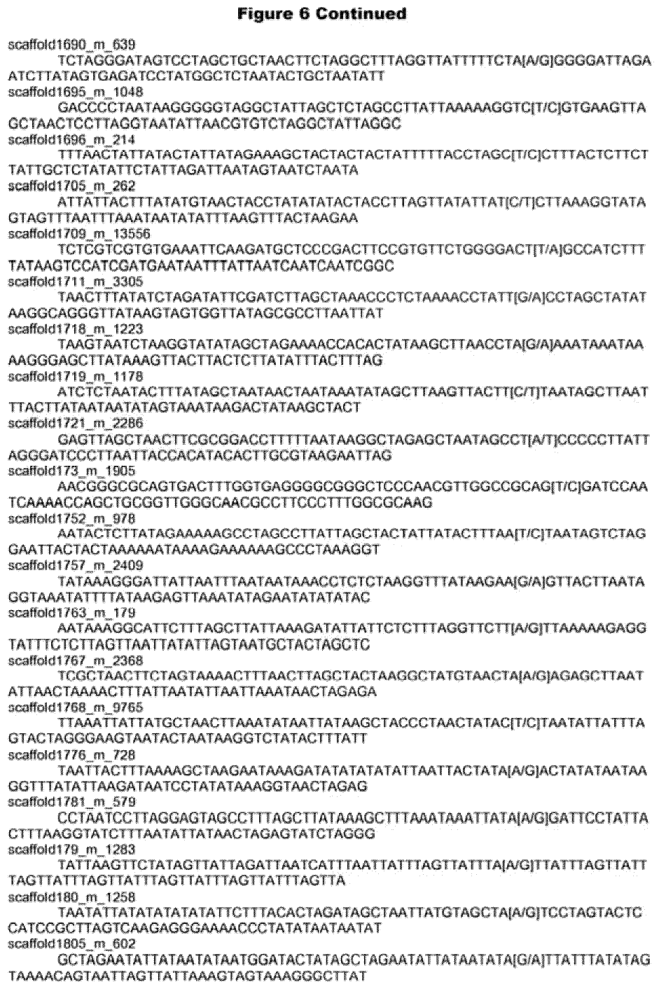

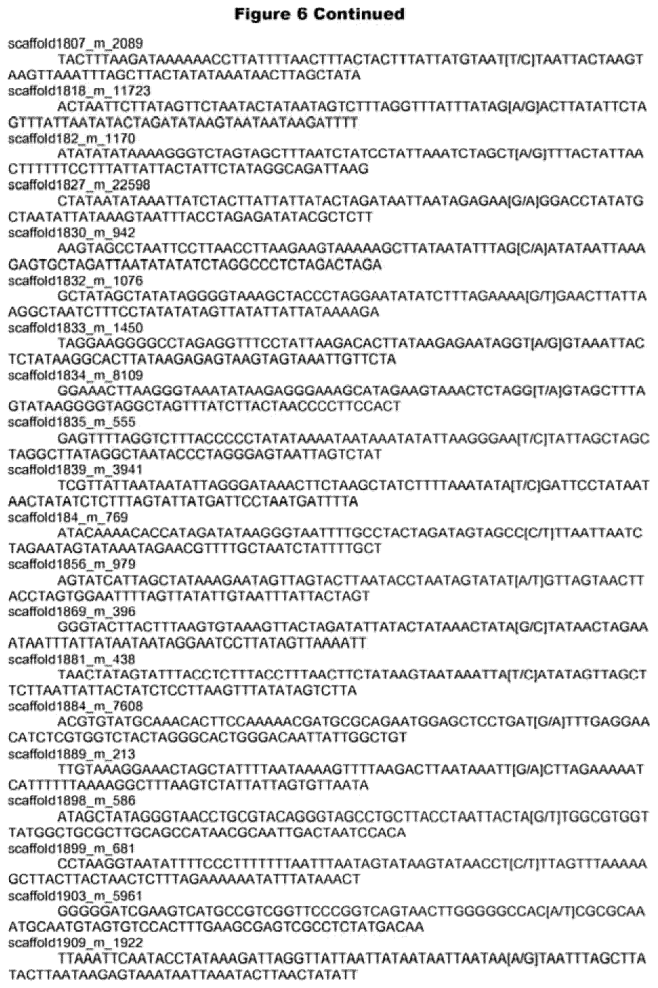

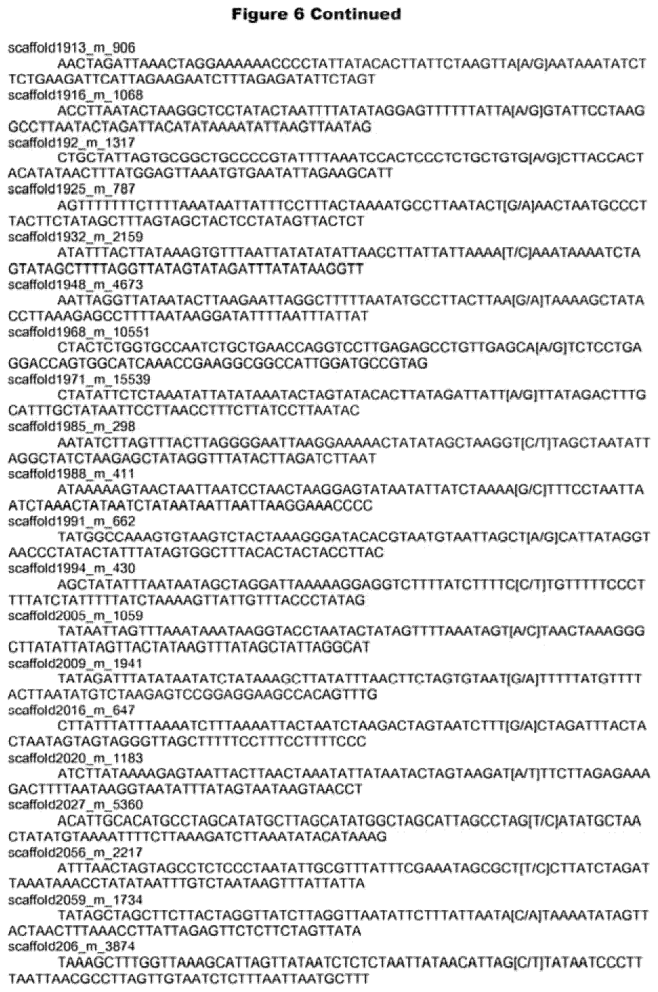

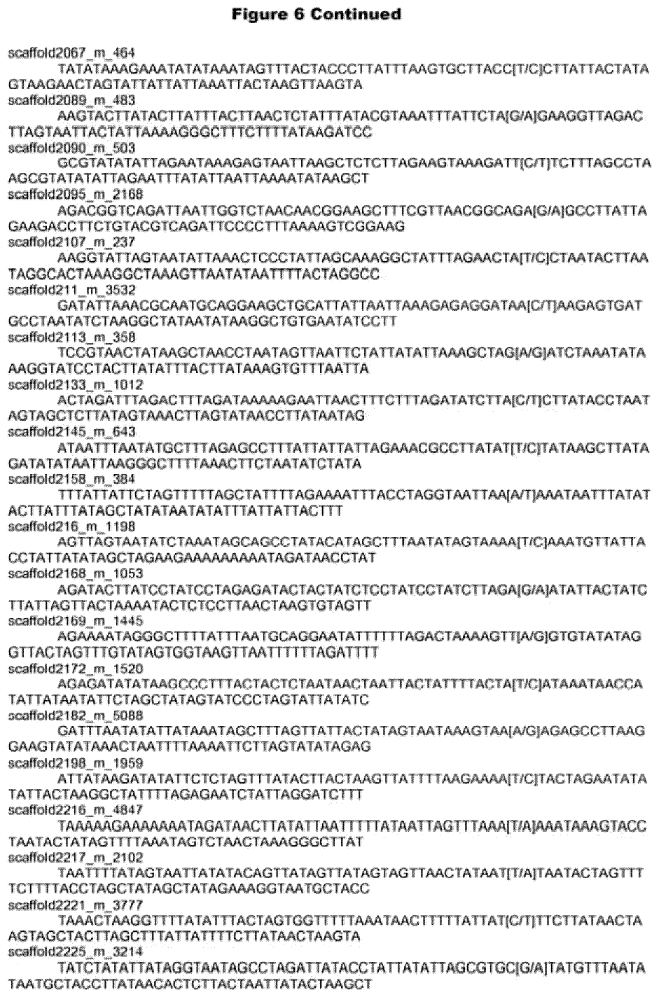

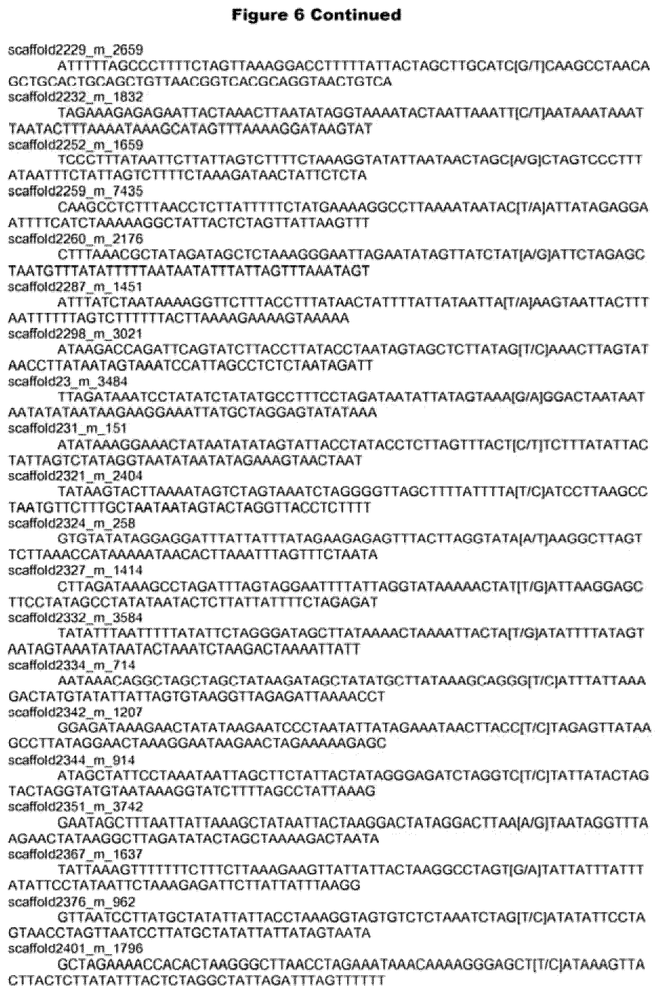

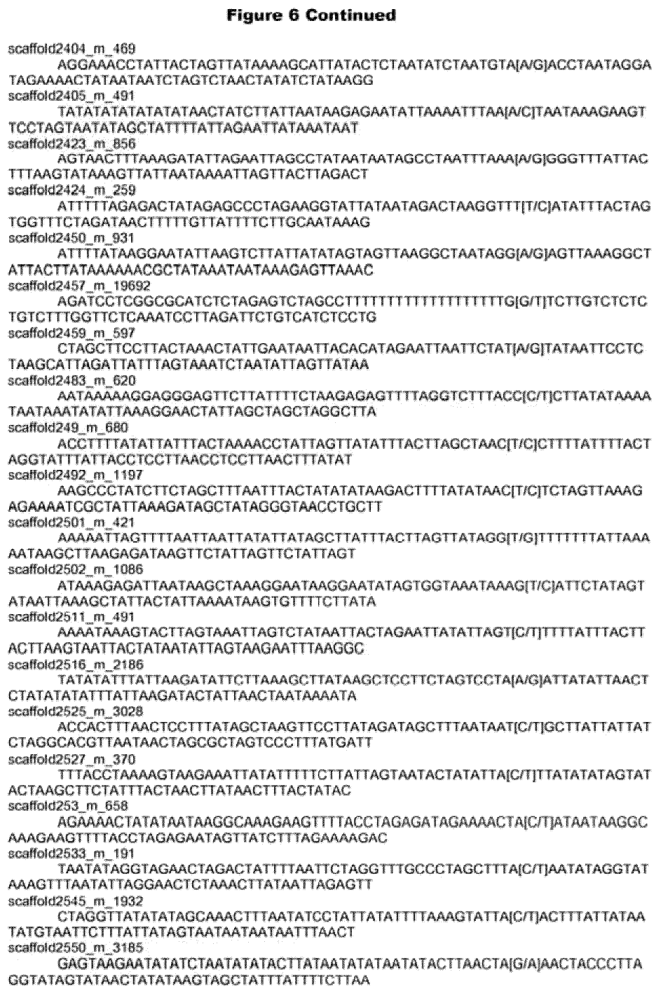

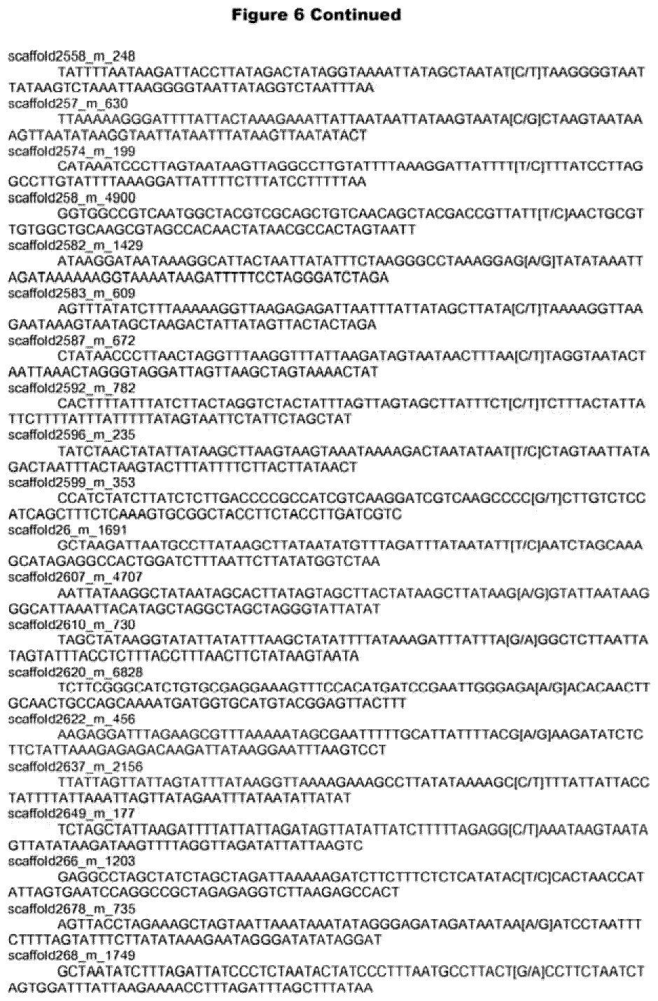

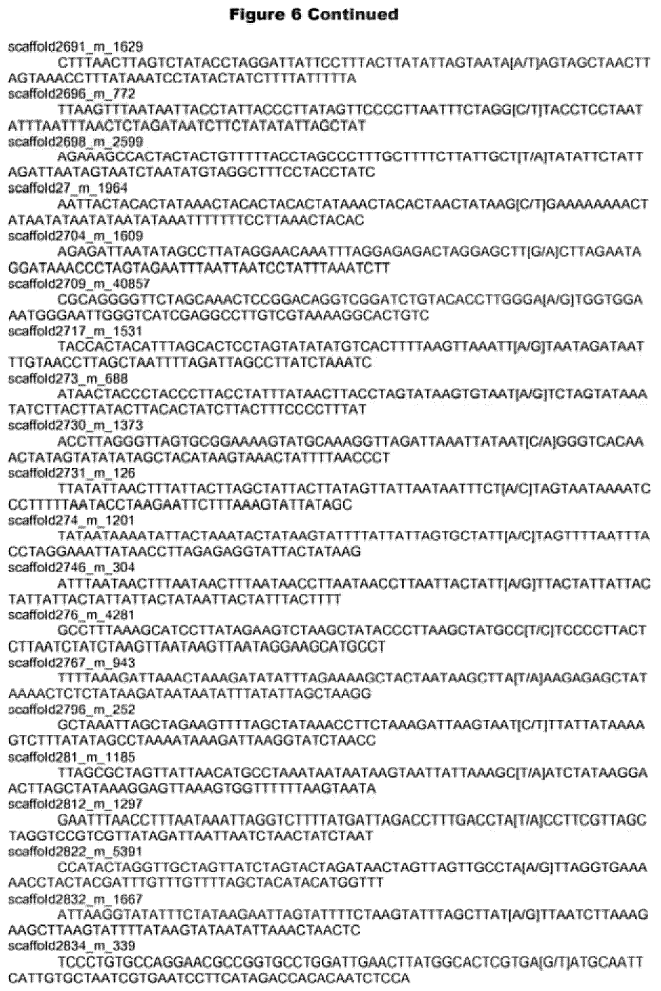

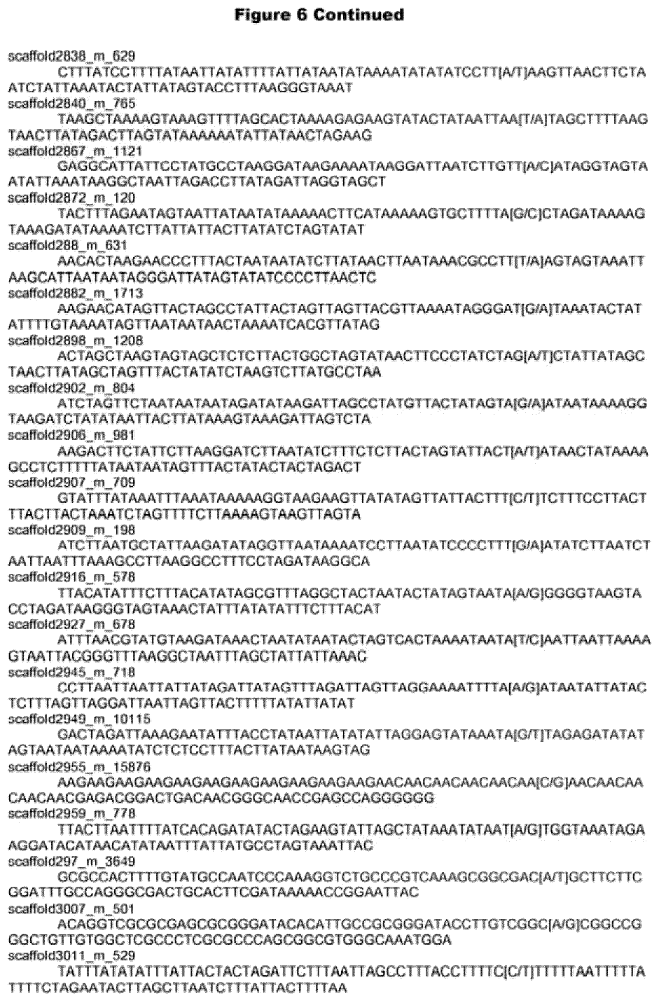

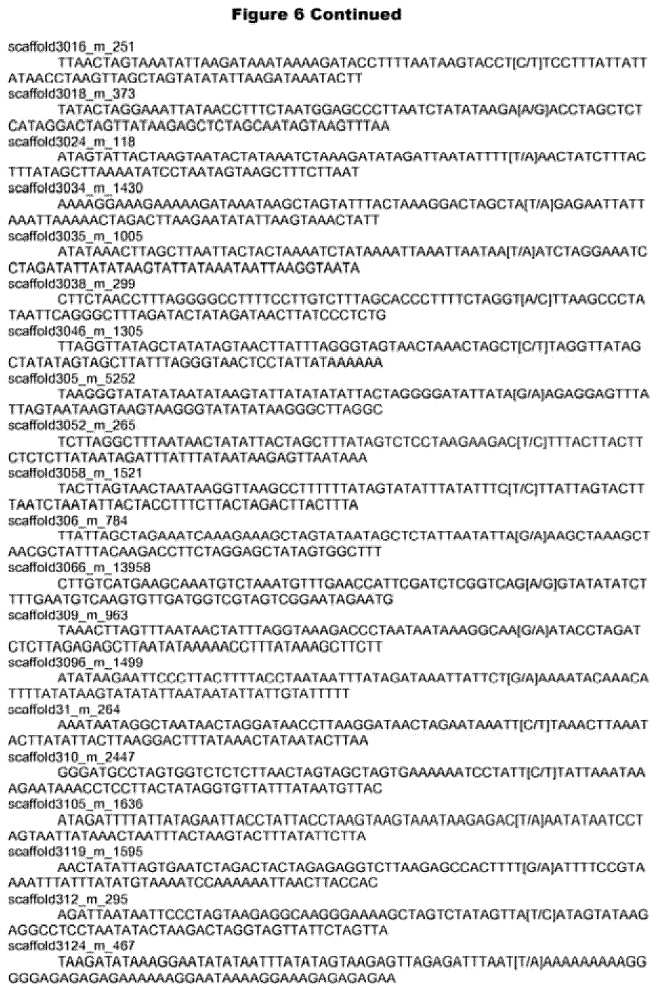

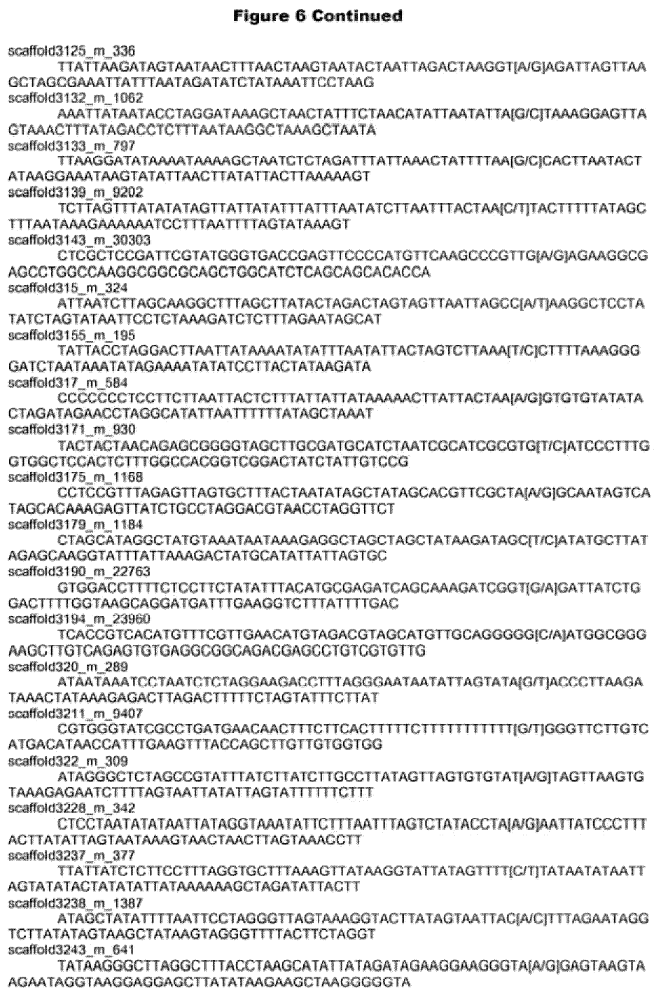

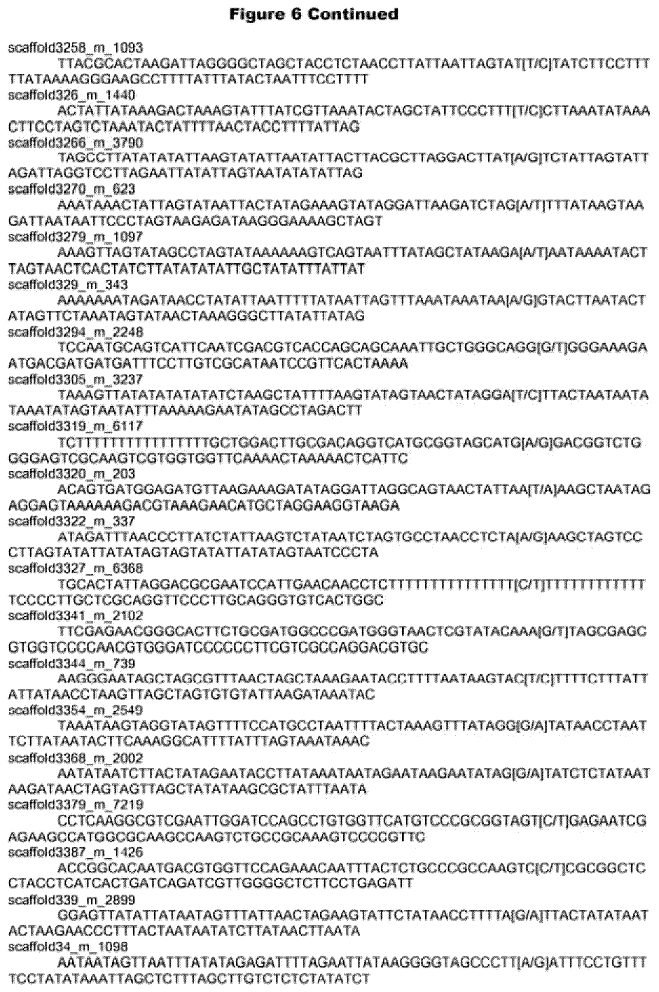

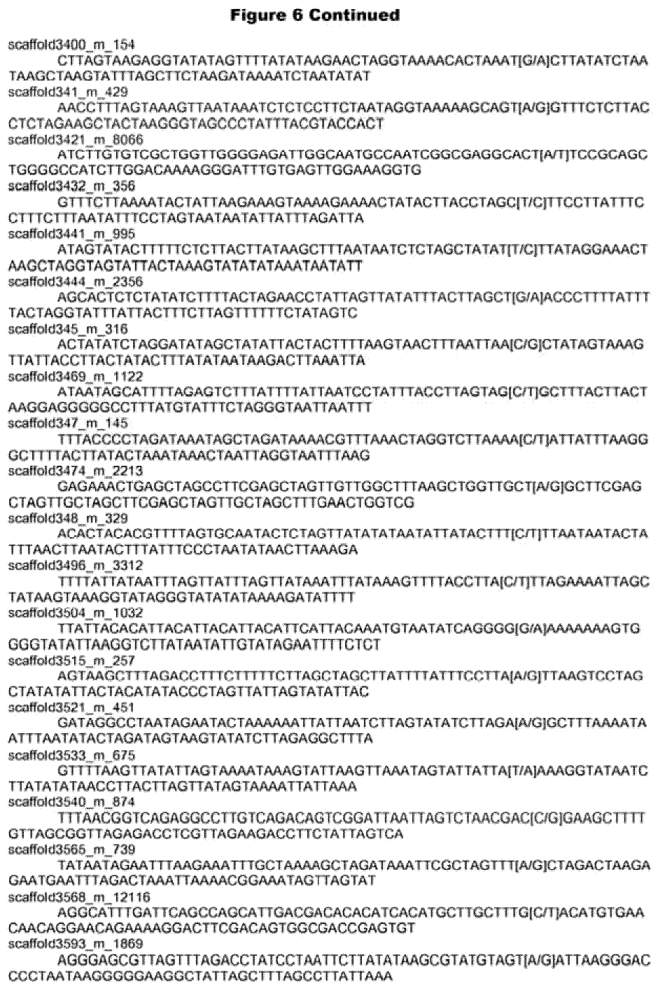

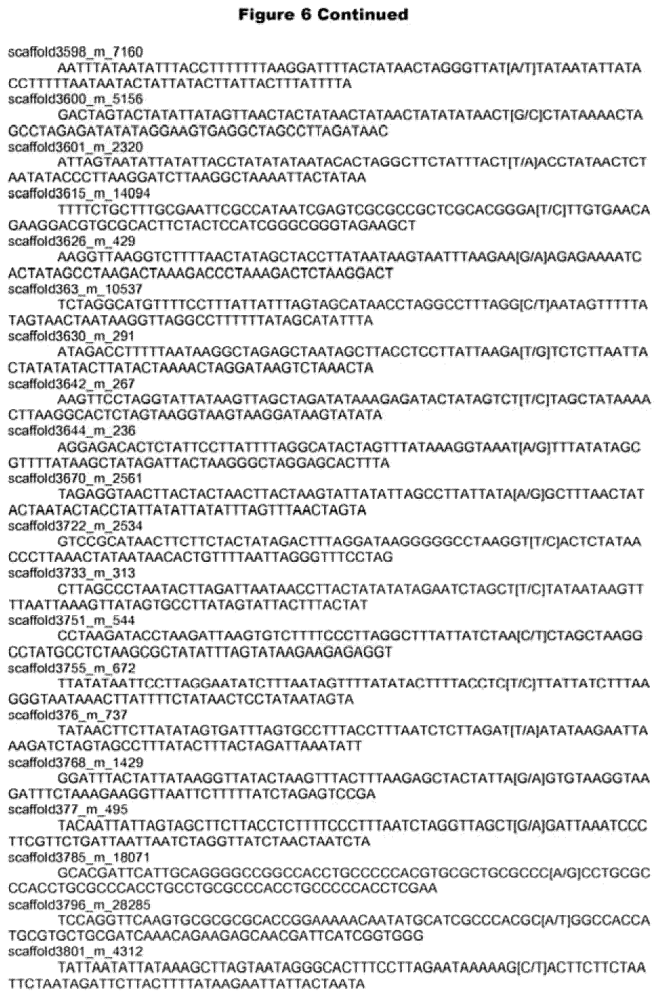

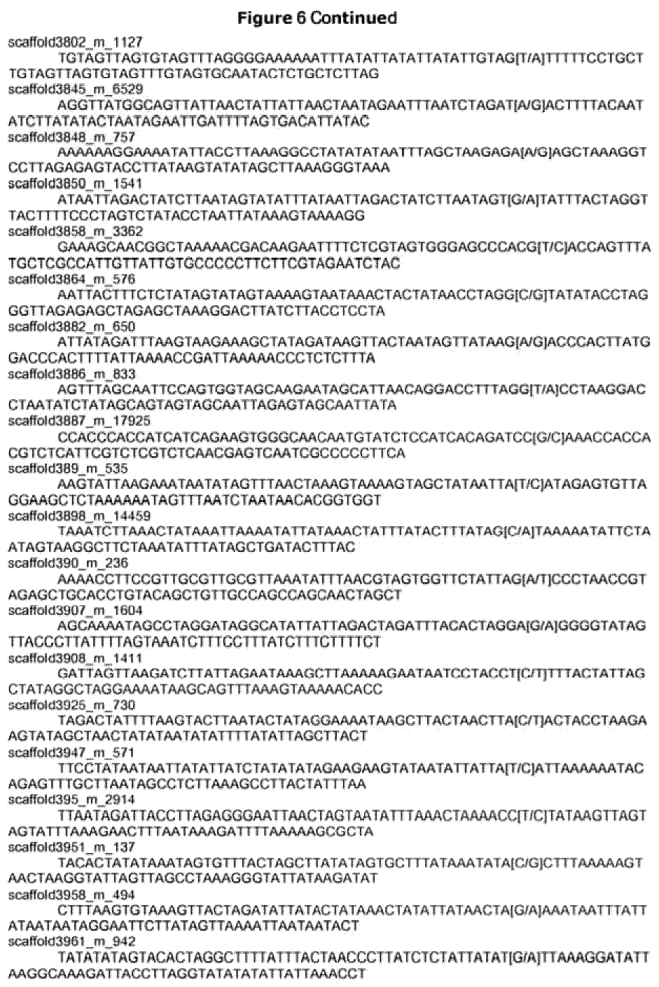

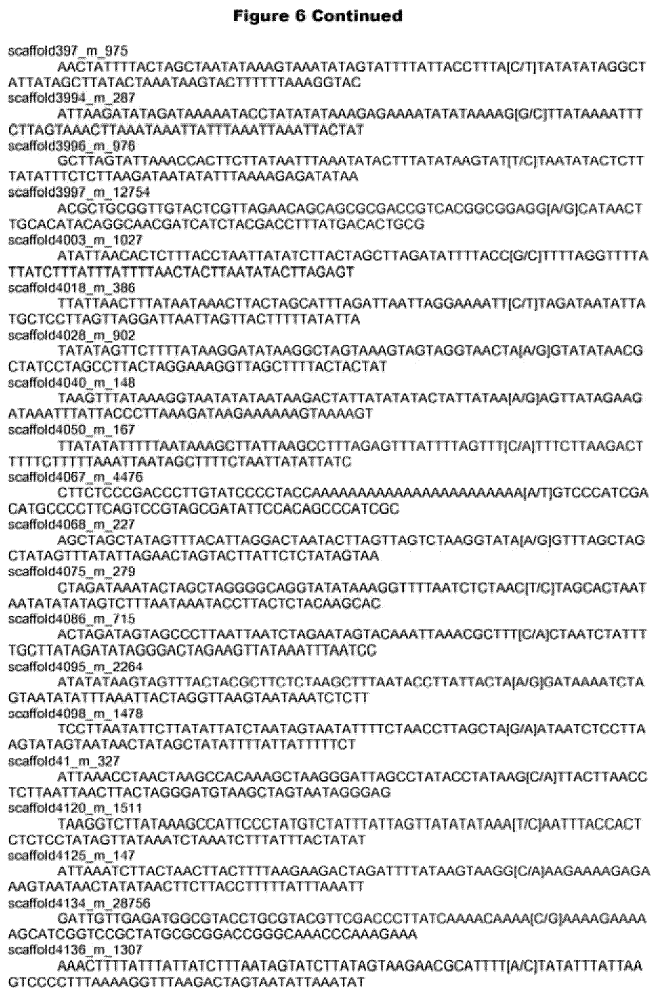

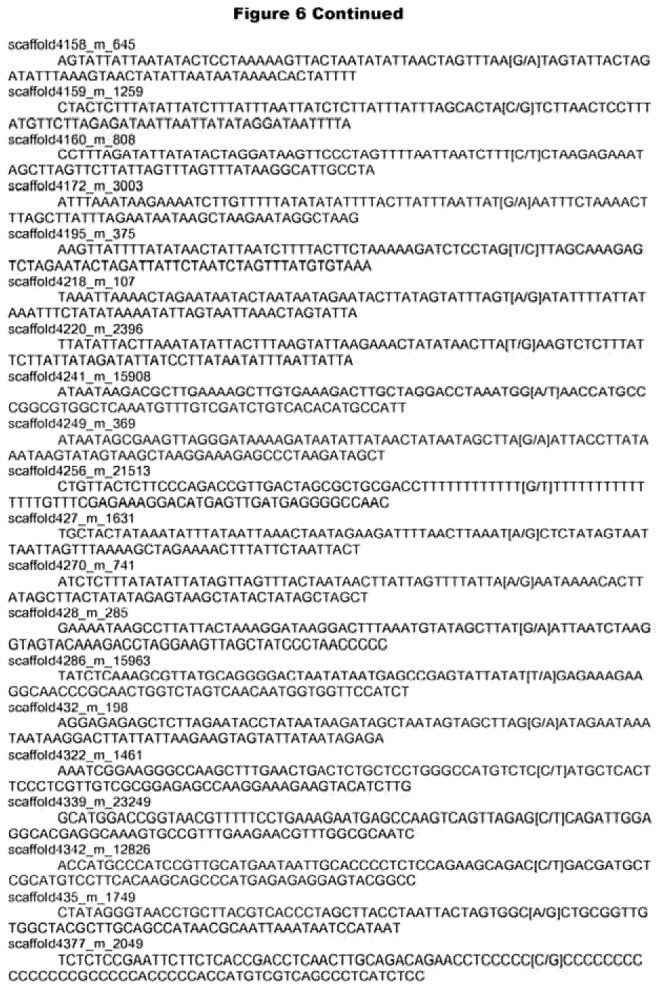

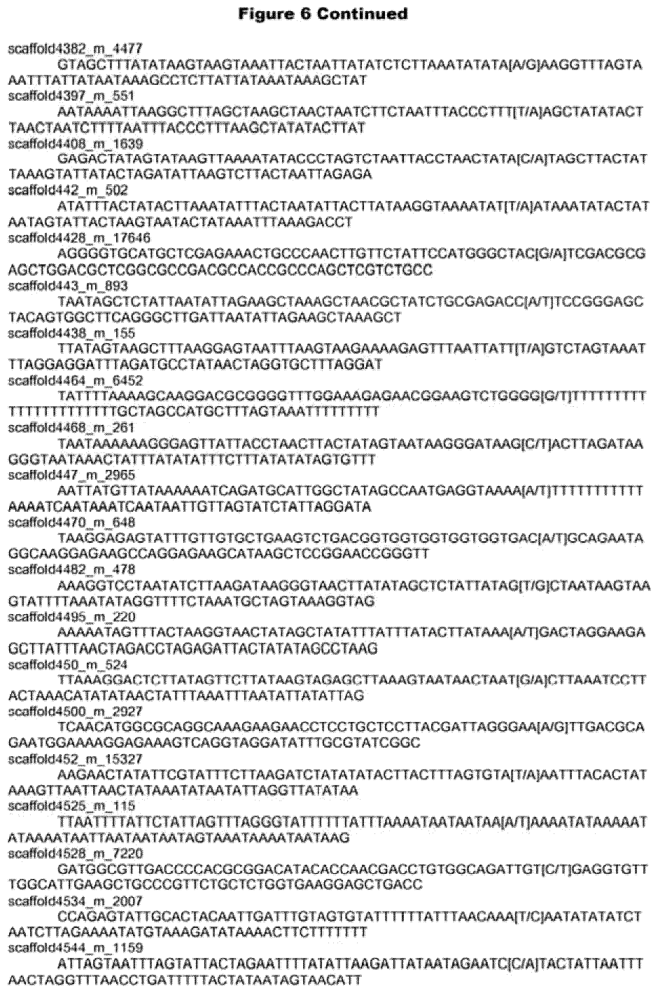

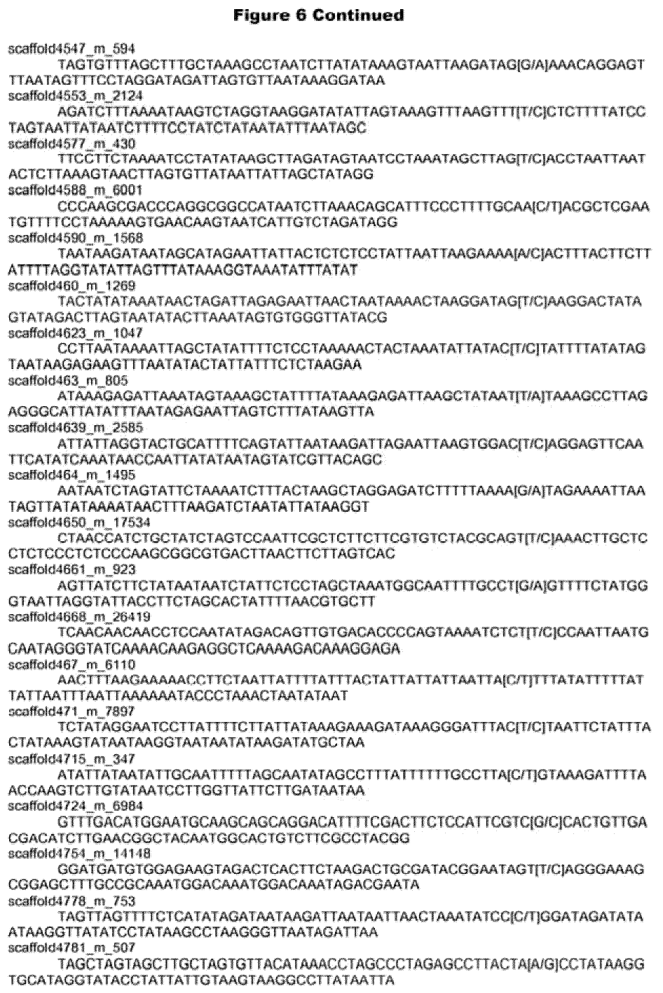

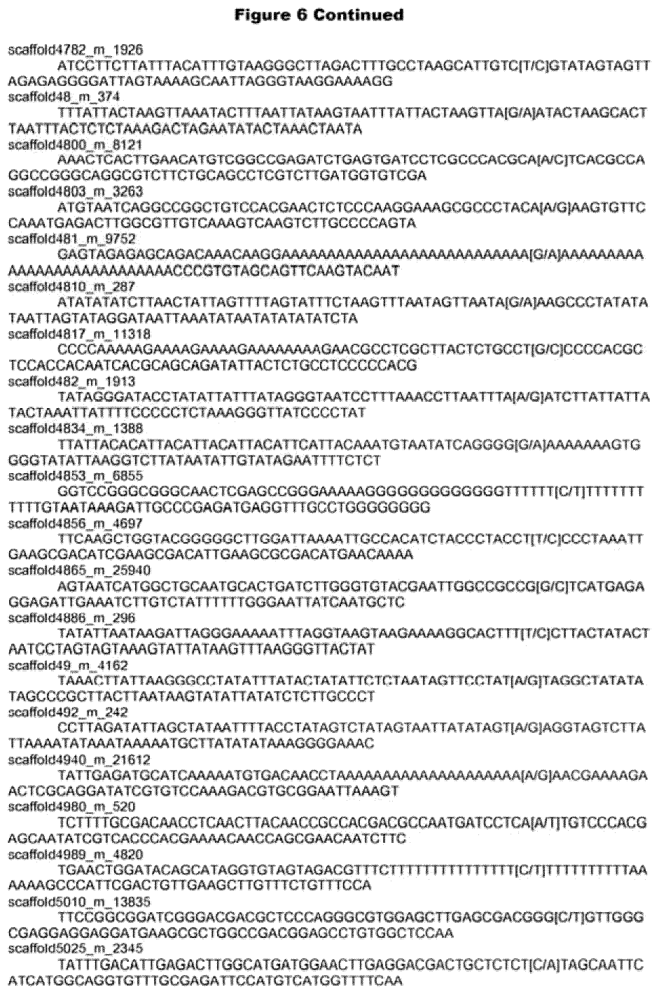

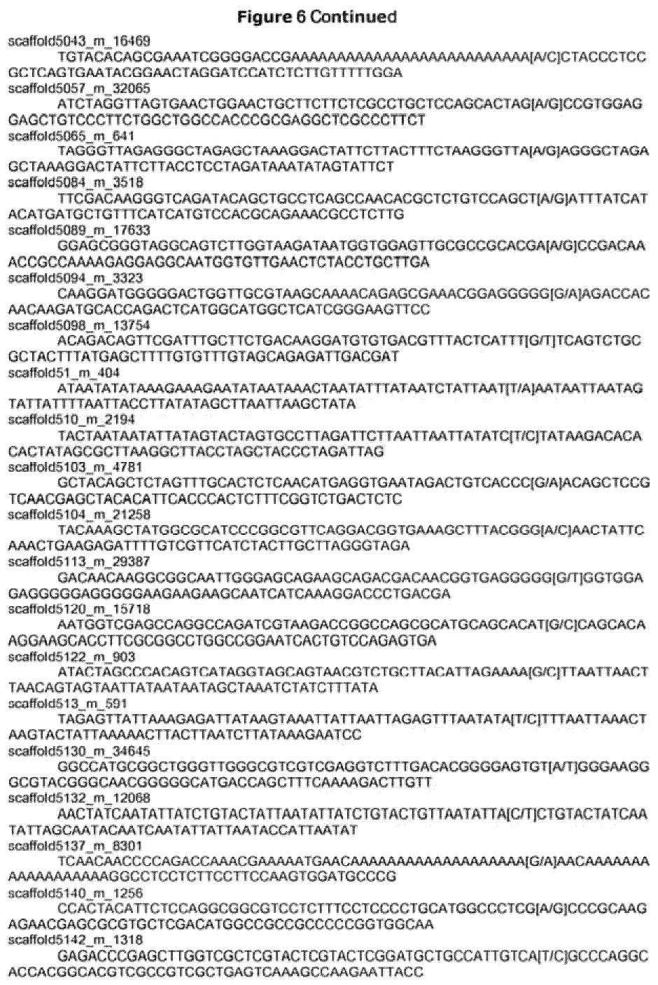

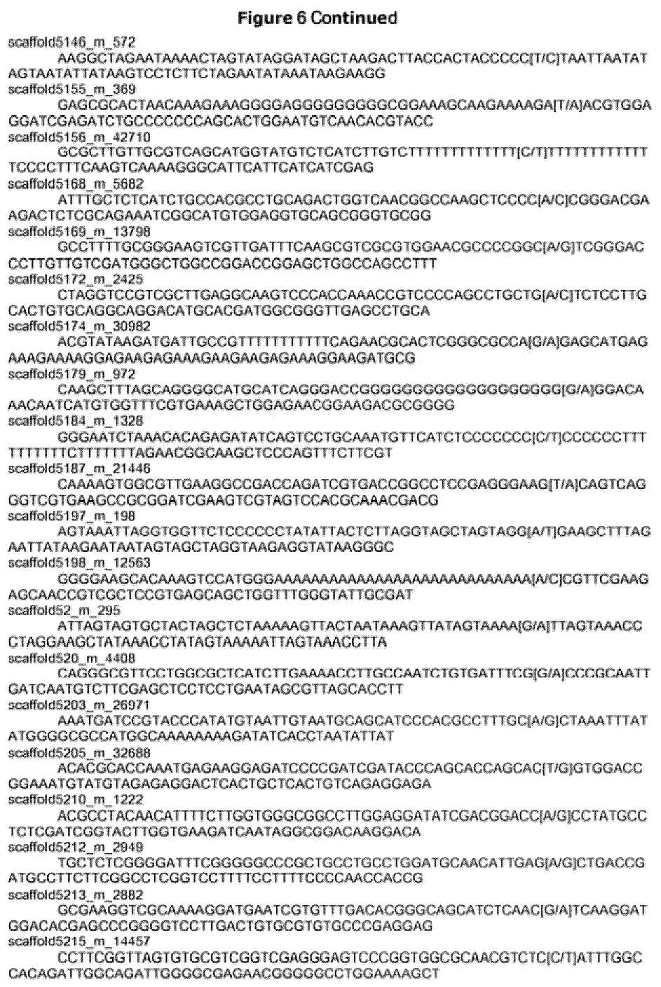

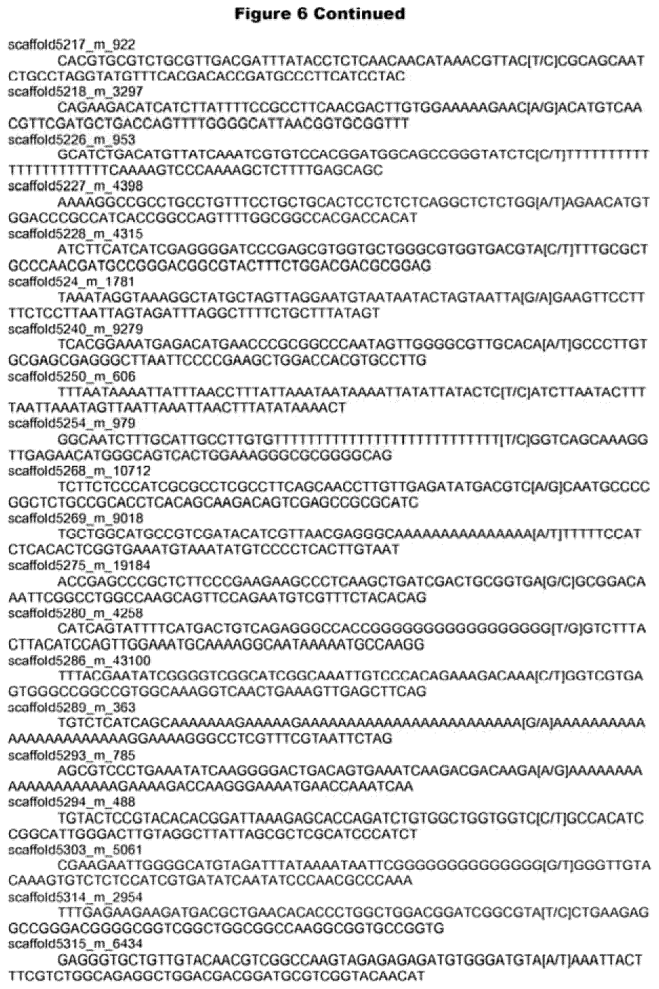

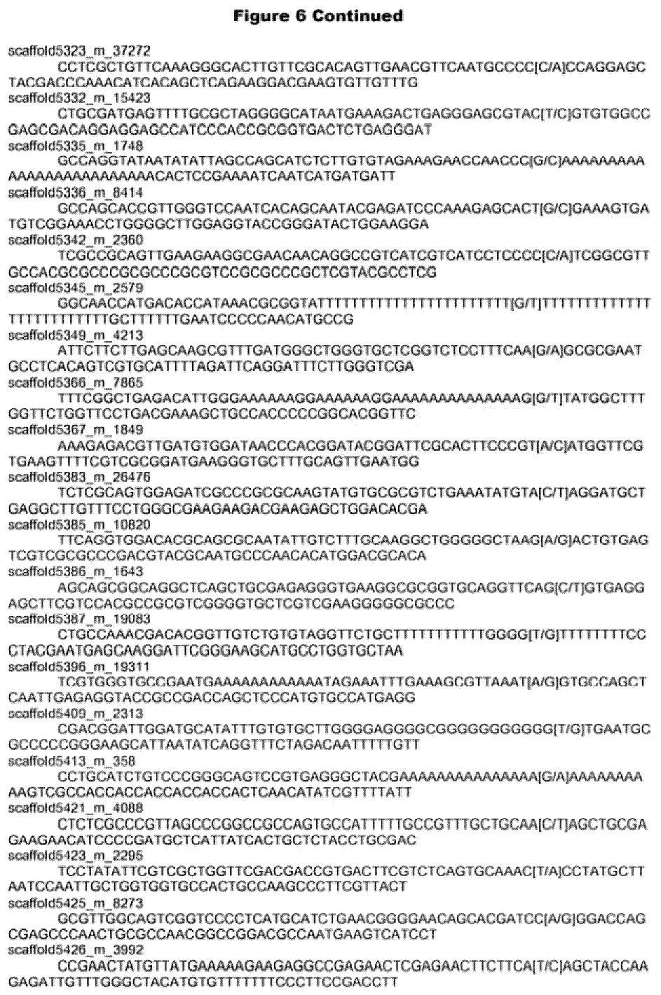

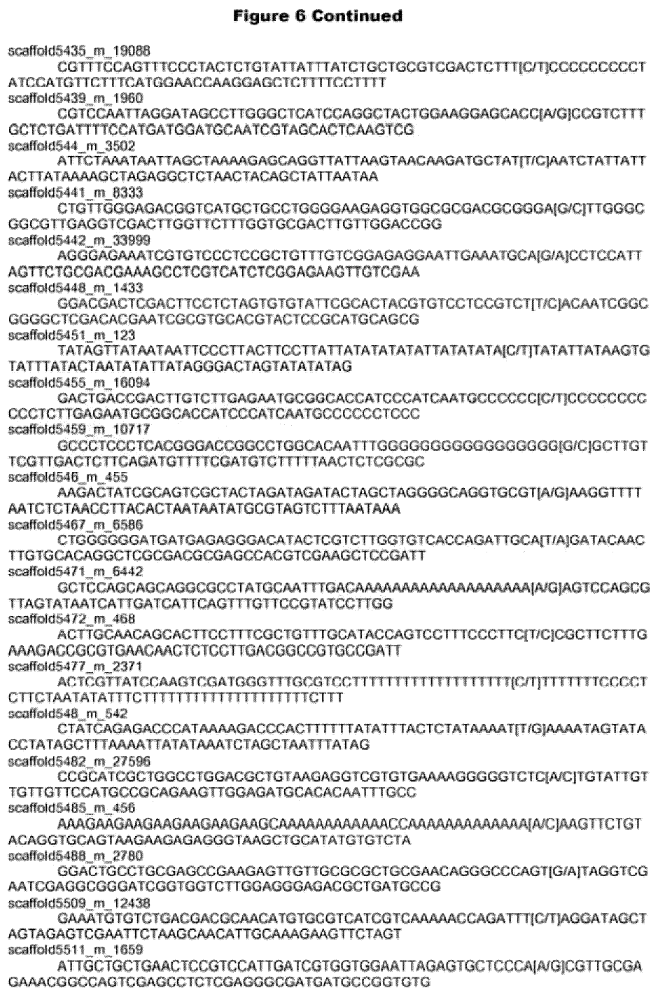

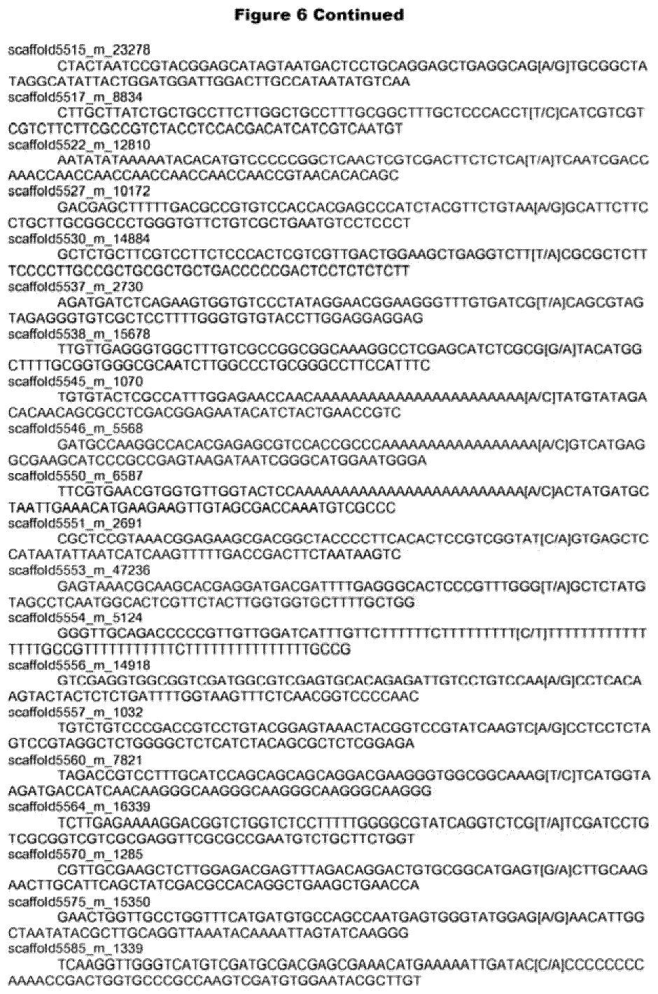

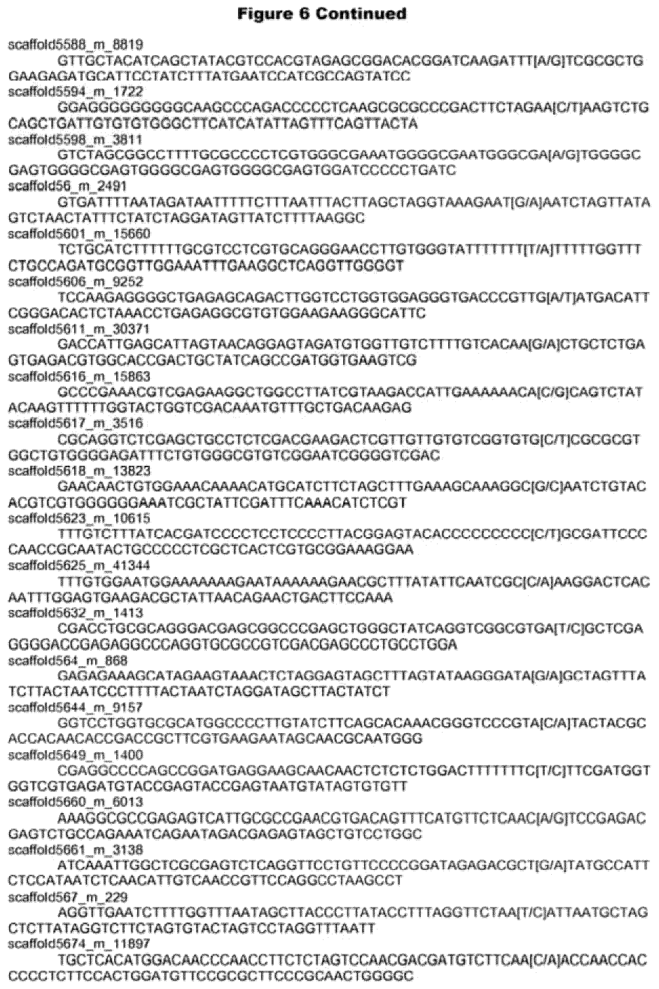

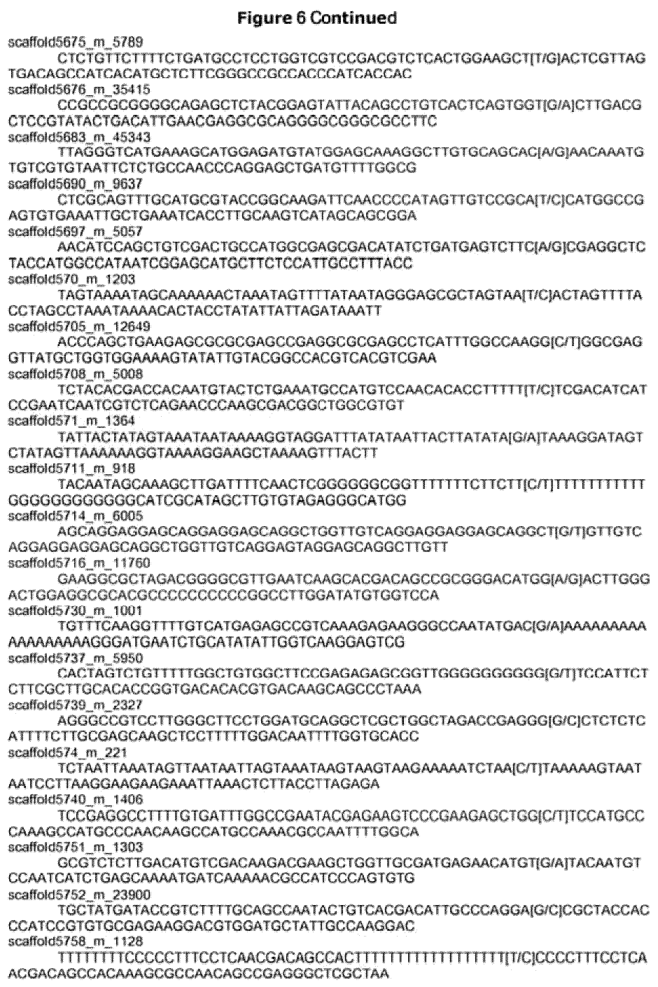

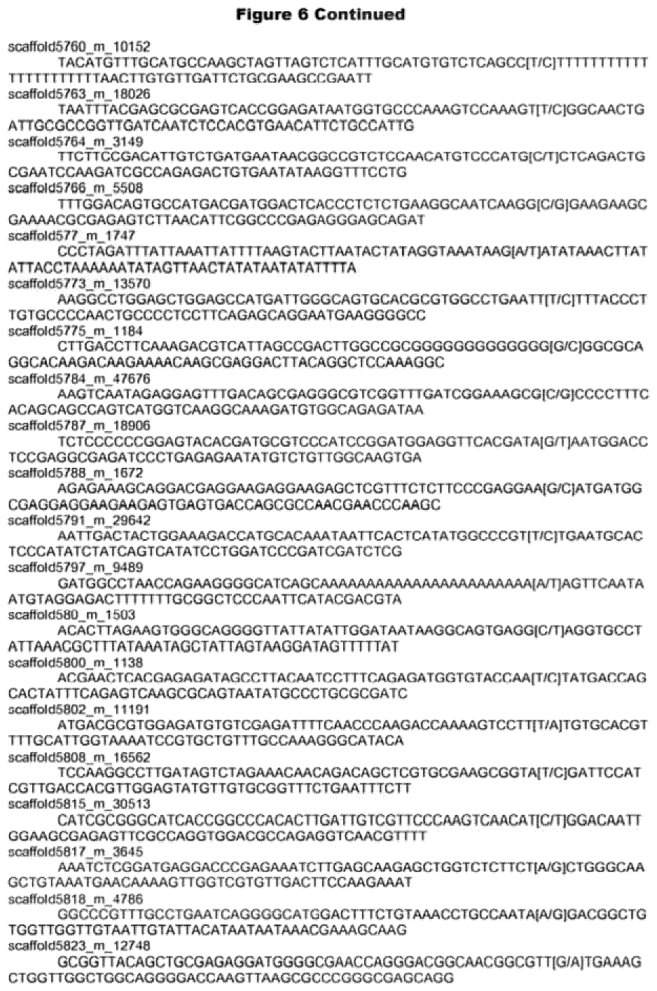

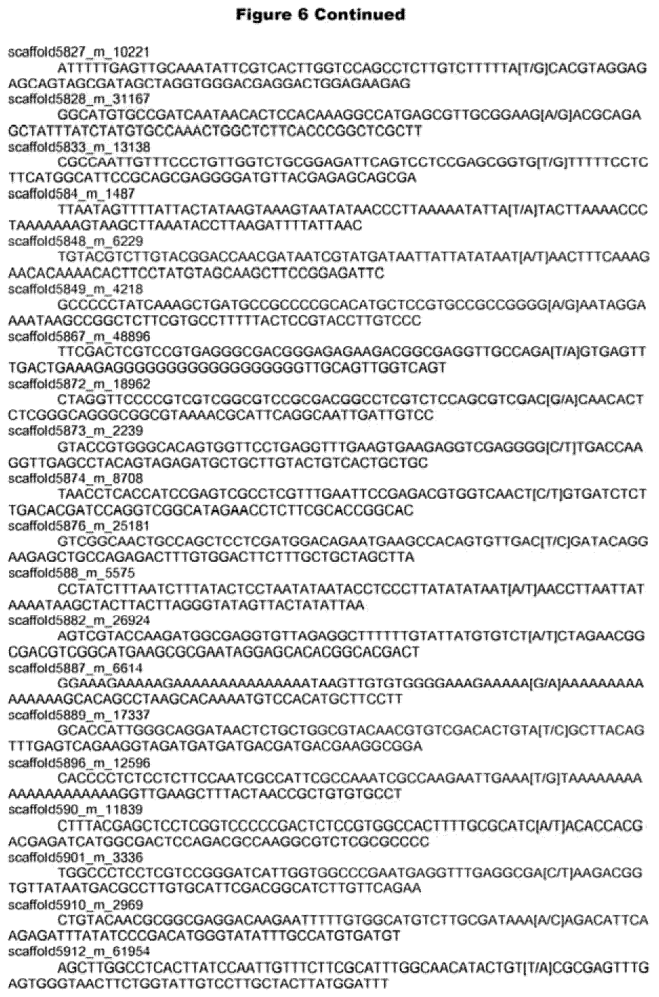

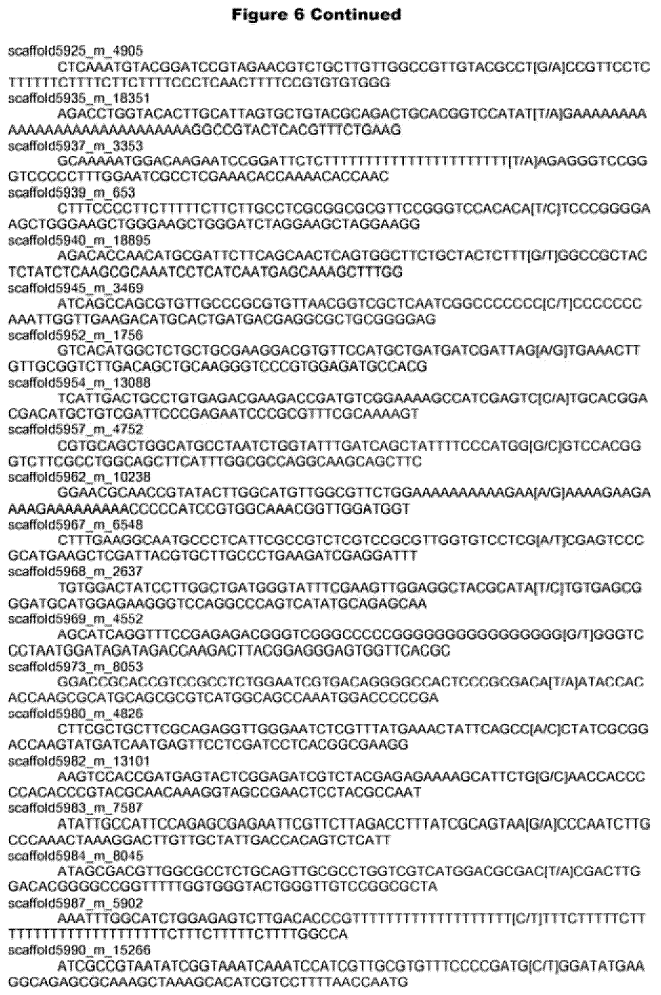

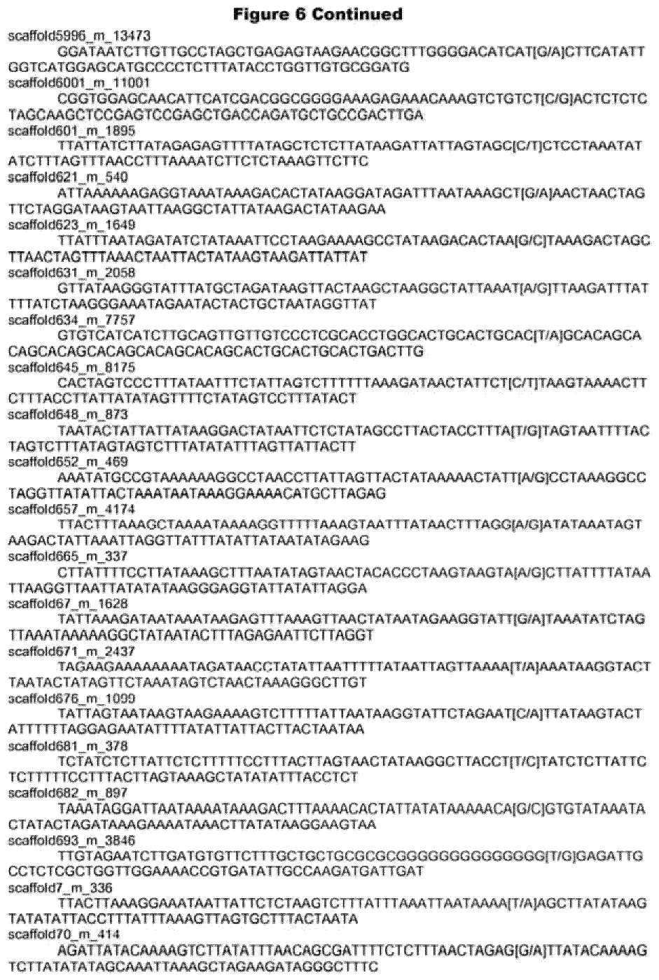

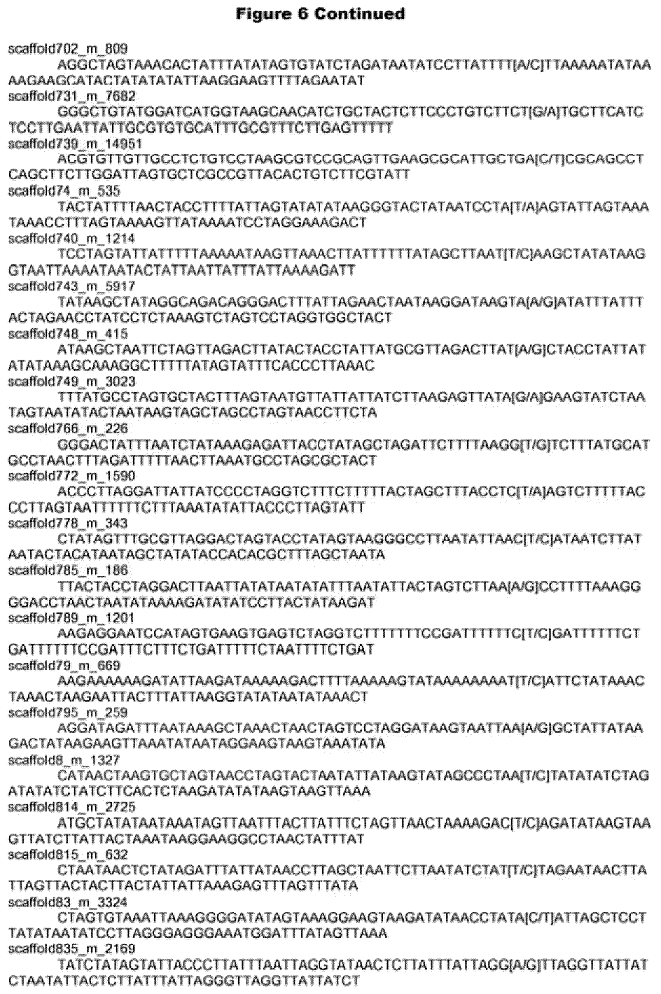

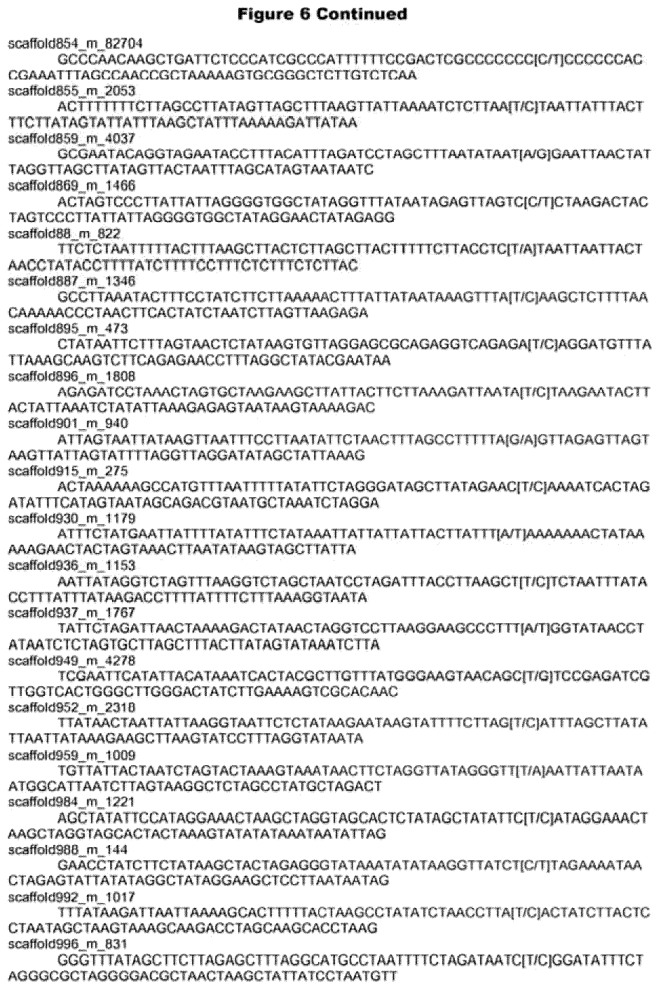

The specific genetic differences of RGT18 genotype over AR37 are detailed in the 817 sequences of the Sequence Listing accompanying this specification and as also depicted in FIG. 6.

### Example 7—Evaluation of Perennial Ryegrass (Lolium perenne) Lines for Resistance/Tolerance to Adult Black Beetle (Heteronychus arator) Feeding

This example details the results of a laboratory screening (choice test) of diploid ryegrass lines to test for resistance/tolerance to adult black beetle (Heteronychus arator) feeding. Black beetle is considered one of the two key pastoral pests in New Zealand along with Argentine stem weevil.

The grass/endophyte combinations (lines) tested were:
RGAS1137 RGT18
SAMSON Nil (control)
SAMSON SE (control).
TROJAN NEA2 (comparator)
SAMSON AR37 (comparator)
RGAS1137 RGT15

Grass tillers were counted on each entry (plant) at Day 0 immediately before the introduction of the adult black beetle. At days 10, 15, 21 post establishment (DPE10, DPE15, DPE21) assessments were made identifying and counting damaged tillers and assessing plant vigour. Plant vigour was scored on a Likert Scale (1 least vigour to 5 most vigour).

Analysis of the results was based on the tiller counts (total, healthy and damaged) and the vigour of plants, being the difference between the Day 0 score and later assessments. Plant vigour is an interpretation of a plant's ability to withstand feeding damage and the effects of adult black beetle test biting.

### Key Results

Plant vigour at DPEO for all lines was assessed as being a 5—(range 1-5).
At DPE10, the damaged tiller count mean for SAMSON Nil (mean 16.4 or 47%) was significantly different (higher) than the balance of the entries (p<0.01). The vigour score mean for SAMSON Nil was 2.6, which was lower than the mean for the balance of the entries (overall mean 4.7). The results for SAMSON Nil were consistent with severe black beetle feeding damage.
At DPE10 the damaged tiller count means for the balance of the entries were not significantly different from each other.
While there were significant differences in mean tiller counts between lines when the assay was started, the results of the assay are conclusive with respect to the poor performance of SAMSON Nil.

The methodologies and results from a study of feeding choices of black beetle (Heteronychus arator) under laboratory conditions is outlined below. The feeding choices were diploid perennial ryegrass (Lolium perennes) varieties containing a range of endophytes (Acremonium loliae). The primary objective of the study was to measure adult black beetle feeding choices.

### Methods

### Adult Black Beetle Collection and Maintenance

In April, 200×75 mm diameter pitfall traps were placed in sheep and beef pasture (with a history of black beetle damage) in Tahuna, Waikato, to trap adult black beetle. The traps were emptied daily until a total of 200 black beetle were collected (90 were required for experimental purposes).

In preparation for the experiment the black beetles were contained individually in cube trays enabling monitoring of individual insects, isolation in case of disease, and preventing combat injury. The beetles were transferred to clean containers every second day and provided with fresh carrot for feeding. The black beetles were maintained at ambient temperatures and not fed for two days prior to entry into the assay.

### Choice Test Set-Up

Six (6) perennial rye grass seedling types of diploid seed-lines and endophyte selections (Table 10) were received from Seed Force, Christchurch, where they had been grown from seed in planter trays. All plants had been tested for endophyte using the tissue print immunoblot method. The plants were well-grown; all lines had large numbers of healthy tillers.

To prepare the experiment representative seedlings of each type (entries) were planted equidistant apart (8 cm) in a sandy loam soil (depth 10 cm), within the margins of galvanised sheet iron ring (height 15 cm diameter 32 cm). Plant position in each ring was randomised, and positions were labelled. There were 15 replications of each entry (i.e. 15 rings). The rings were covered with insect netting suspended 15 cm above the soil surface.

TABLE 10
Entries into adult black
beetle feeding assay
Entry
RGAS1137 RGT18
SAMSON Nil
SAMSON SE
TROJAN NEA2
SAMSON AR37
RGAS1137 RGT15

The experiment was maintained in a screen house at ambient temperatures and checked daily to ensure the soil moisture was adequate and that insect containment integrity was maintained.

### Assessments

The tiller counts and baseline vigour scores for each plant were recorded prior to the introduction of the adult black beetles (Day 0, Assessment 1). The vigour score is a relative score for all plants in the trial on a Likert Scale of 1 (least vigour) to 5 (most vigour). Vigour characteristics were growth form, growth level, leaf colour, and appearance. The plants were then trimmed to 3 cm and adult black beetle were released into each ring (replicate) at a ratio of one per test plant (6 black beetles per ring).

Plants were inspected daily for black beetle adult feeding damage and four assessment made (Table 11) with a final assessment (Day 21, Assessment 4).

TABLE 11
Assessment type and timing post establishment
(introduction of black beetle)

| | Days post | |
|---|---|---|
| Assessment | Establishment (DPE) | Assessment type |
| 1 | 0 | Vigour score, tiller count |
| 2 | 10 | Vigour score, damaged |
| | | tiller count |
| 3 | 15 | Vigour score, damaged |
| | | tiller count |
| 4 | 21 | Vigour score, damaged tiller |
| | | count, confirm tiller count |

### Results

### Initial Tiller Count (DPE0)

A tiller count prior to the introduction of the black beetle adults provided a baseline. The tiller counts ranged from 10 to 52, with variation across the grass/endophyte combinations (lines) (Table 12). The mean range of was from 13.6 to 35.1.

TABLE 12
Baseline tiller count

| | RGAS1137 | SAMSON | SAMSON | TROJAN | SAMSON | RGAS1137 |
|---|---|---|---|---|---|---|
| Line | RGT18 | Nil | SE | NEA2 | AR37 | RGT15 |
| Min | 14 | 21 | 14 | 10 | 12 | 4 |
| Max | 45 | 52 | 38 | 27 | 34 | 21 |
| Median | 31 | 33 | 26 | 15 | 21 | 15 |
| Mean | 28.0 | 35.1 | 25.9 | 16.3 | 21.9 | 13.6 |

Ideally the mean number of tillers in each of the lines is sufficiently similar that they could statistically be from the same population. Table 12 and FIG. 7 show the mean baseline tiller count at day zero for each grass/endophyte combination. An ANOVA of tiller counts at planting (Day 0) confirmed that there was a significant difference between the means (p<0.01). T tests showed lines were split into four significantly different groups. SAMSON Nil had significantly higher mean than the rest of the lines. The statistical groupings based on the means were RGAS1137 RGT18 and SAMSON SE; SAMSON SE and SAMSON AR37; and TROJAN NEA2 and RGAS1137 RGT15. These differences are discussed below.

### Day Zero Initial Vigour Score (DPE0)

All entries achieved a vigour score of 5 prior to the introduction of the black beetle adults.

### Day 10 Post Establishment (DPE10)

Plants were tended regularly and visually checked for black beetle damage following planting. On Day 10 there was sufficient damage to undertake a tiller count and vigour scoring.

### Tiller Damage

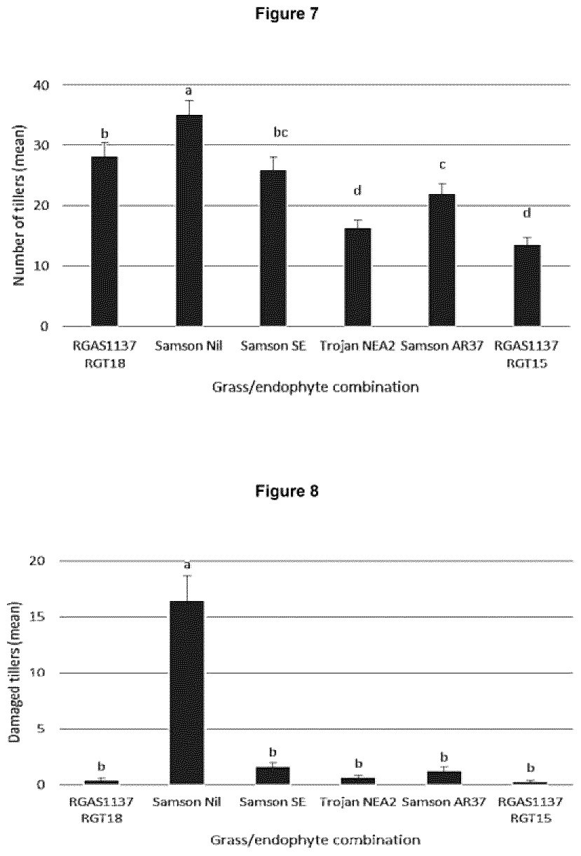

At DPE10 damaged tillers were counted and the mean for each calculated-refer Table 13 and FIG. 8. An ANOVA of damaged tillers at Day 10 confirmed a difference in the means at a 1% level of significance (p<0.01). T-tests showed that the mean damaged tillers for SAMSON Nil was significantly different, and higher than all other lines.

The mean number of damaged tillers for SAMSON Nil was 16.4 (range 5-30) (Table 13). The next highest level of damage was SAMSON SE, with a mean of 1.6 (range 0-5). The line RGAS1137-RGT15 had the least damage with a mean of 0.2 (range 0-3). The line RGAS1137-RGT18 was similar.

TABLE 13
DPE10 damaged tiller count

| | RGAS1137 | SAMSON | SAMSON | TROJAN | SAMSON | RGAS1137 |
|---|---|---|---|---|---|---|
| Line | RGT18 | Nil | SE | NEA2 | AR37 | RGT15 |
| Min | 0 | 5 | 0 | 0 | 0 | 0 |
| Max | 3 | 30 | 5 | 2 | 5 | 3 |
| Median | 0 | 15 | 1 | 0 | 0 | 0 |
| Mean | 0.3 | 16.4 | 1.6 | 0.6 | 1.2 | 0.2 |

T-tests of the means of damaged tillers showed that the mean for SAMSON Nil was statistically different from the means for all other lines. The means for all other lines were statistically the same.

Because SAMSON Nil had a significantly greater number of tillers at DPEO and had the greatest mean number of damaged tillers a question arose as to whether there was any significance in this relationship. Testing the number of damaged tillers as a proportion of the tiller count at planting confirmed that as a proportion of total tillers at DPEO, SAMSON Nil had a significantly greater mean than other lines (Table 14). The mean proportion across the plots for SAMSON Nil was 0.50, followed by SAMSON SE (0.074), SAMSON AR37 (0.054), TROJAN NEA2 (0.035), RGAS1137 RGT15 (0.011), and RGAS1137 RGT18 (<0.00). Therefore, considering the higher starting number of tillers, we conclude that the mean damaged tillers for SAMSON Nil is statistically different from all other lines.

TABLE 14
Proportion of damaged tillers at DPE10

| | RGAS1137 | SAMSON | SAMSON | TROJAN | SAMSON | RGAS1137 |
|---|---|---|---|---|---|---|
| Line | RGT18 | Nil | SE | NEA2 | AR37 | RGT15 |
| Proportion | <0.00 | 0.50 | 0.074 | 0.035 | 0.054 | 0.011 |
| damaged | | | | | | |

### Vigour Scores

The vigour score is a Likert Scale with 1 is least vigorous and 5 is most vigorous. A zero score represents a dead plant. At the initial planting (DPE0) all plants included in the assay were assessed as having a vigour score of 5.

At DPE10, SAMSON Nil recorded the lowest mean vigour score of 2.6. No dead plants were recorded—refer Table 15.

TABLE 15
Vigour scores at DPE10

| | RGAS1137 | SAMSON | SAMSON | TROJAN | SAMSON | RGAS1137 |
|---|---|---|---|---|---|---|
| Line | RGT18 | Nil | SE | NEA2 | AR37 | RGT15 |
| Min | 4 | 1 | 3 | 4 | 3 | 4 |
| Max | 5 | 4 | 5 | 5 | 5 | 5 |
| Median | 5 | 3 | 5 | 5 | 5 | 5 |
| Mean | 4.9 | 2.6 | 4.4 | 4.7 | 4.5 | 4.9 |

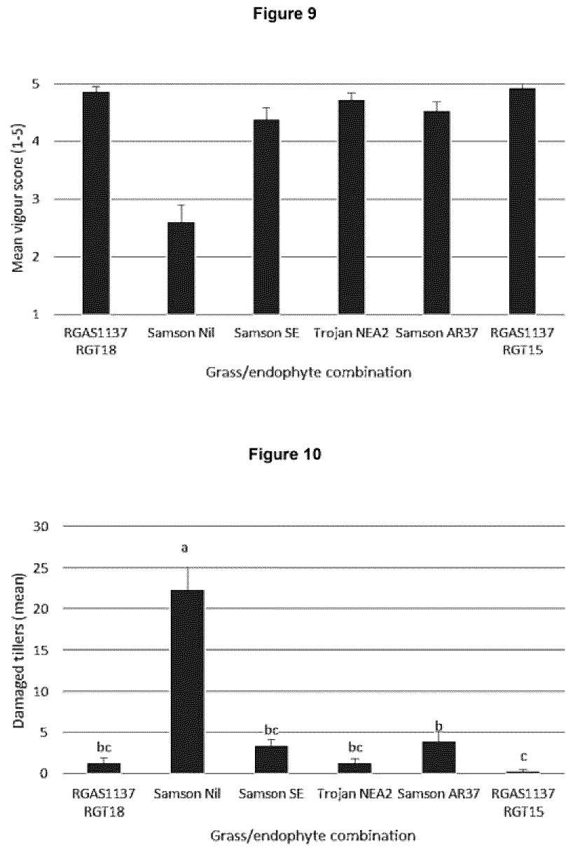

Except for SAMSON Nil (mean vigour score 2.6) the vigour scores (with SE) indicate a high level of vigour across all lines at DPE10 (refer Table 15/FIG. 9).

TABLE 16
Mean damaged tiller counts at DPE10, DPE15, DPE21

| | RGAS1137 | SAMSON | SAMSON | TROJAN | SAMSON | RGAS1137 |
|---|---|---|---|---|---|---|
| DPE | RGT18 | Nil | SE | NEA2 | AR37 | RGT15 |
| 10 | 0.3 | 16.4 | 1.6 | 0.6 | 1.2 | 0.2 |
| 15 | 1.3 | 22.3 | 3.5 | 1.3 | 3.9 | 0.3 |
| 21 | 2.0 | 18.3 | 5.8 | 1.9 | 7.1 | 0.4 |

At DPE15 (refer Table 16/FIG. 10) there was a statistical difference (ANOVA) in the mean number of damaged tillers across the lines (p<0.001). T-tests showed a greater spread than at Day 10, with RGAS1137-RGT15 having a significantly fewer damaged tillers (mean) than all other lines.

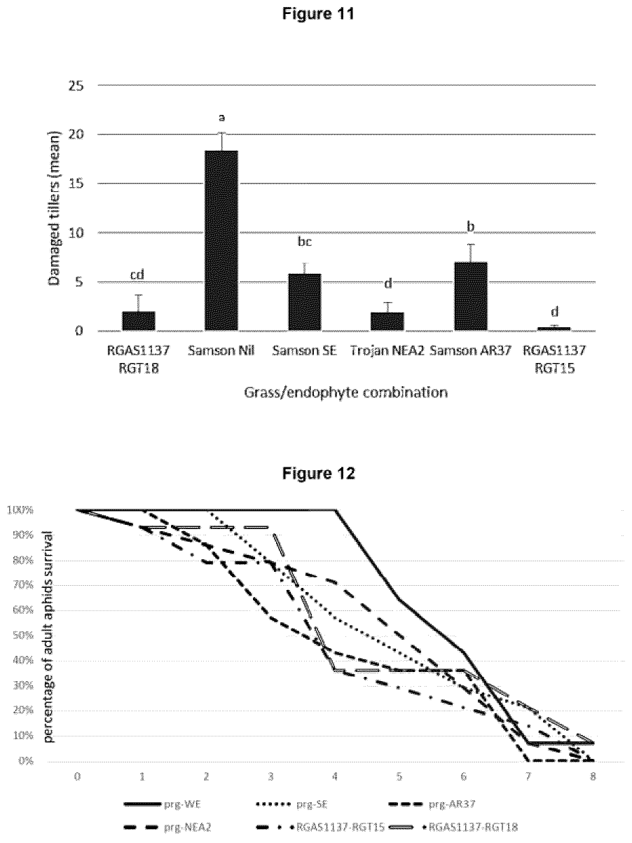

At DPE21 (refer Table 16/FIG. 11) there was a statistical difference (ANOVA) in the mean number of damaged tillers across the endophyte lines (p<0.001). T-tests showed a greater spread than at Day 21, with the lines now split into four significantly different groups

Testing the mean damaged tillers as a proportion of the tiller count at planting showed that, while the order remains the same, the proportion of damaged tillers for SAMSON Nil had decreased as a proportion of tillers at planting (0.587). This was attributed to due to the recovery of some damaged tillers as black beetle feeding pressure lessened.

TABLE 17
Summary of mean vigour scores at DPE10, DPE15, DPE21

| | RGAS1137 | SAMSON | SAMSON | TROJAN | SAMSON | RGAS1137 |
|---|---|---|---|---|---|---|
| DPE | RGT18 | Nil | SE | NEA2 | AR37 | RGT15 |
| 10 | 4.9 | 2.6 | 4.4 | 4.7 | 4.5 | 4.9 |
| 15 | 4.6 | 2.1 | 3.7 | 4.4 | 3.9 | 4.8 |
| 21 | 4.7 | 2.0 | 3.5 | 4.3 | 3.6 | 4.9 |

With the exception of RGAS1137 RGT18 and RGAS1137 RGT15 which recorded increases in vigour scores of between DPE 15 and 21, the balance of the entries recorded declines. The vigour score for SAMSON Nil reflects continued feeding attention by the black beetle, despite the fact that 64% (range 10%-90%) of tillers were damaged.

### Conclusion

Presence of either endophyte RGT15 or RGT18 in host grasses was found to confer better resistance/tolerance to black beetle (Heteronychus arator) feeding compared to nil endophyte varieties.

The DPE21 results indicate that both RGT15 and RGT18 endophytes confer a better vigour score and thus are more resistant to insect feeding compared to SAMSON Nil, SAMSON SE, TROJAN NEA2 and importantly provide a significant improvement over SAMSON AR37.

### Example 8-Evaluation of Presence of RGT15 and RGT18 Infected Varieties of Ryegrass on the Presence of Root Aphid

Methods

The effect of perennial ryegrass endophyte on the mortality and fecundity of the mealy grass root aphid, Aploneura lentisci Passerini (Homoptera, Aphididae), was examined using an in vitro bioassay.

### Aphid Diet Preparation

Roots from a pooled sample of c. 100 12-day old seedlings were ground to a fine powder in liquid nitrogen and suspended in a 20% sucrose/ultrapure water solution. A pooled sample reduces symbiotum-symbiotum variation and provides an indication of the population average.

### Root Aphid Bioassays

Colonies of root aphids were reared on mature endophyte-free perennial ryegrass plants, in a controlled environment room (CER) maintained at 20±2° C. and 62±5% RH, with a photoperiod of 14 h light and 10 h dark.

Single adult aphids were placed in 35 mm petri dishes. A 200 μl aliquot of the diet was sandwiched between two layers of parafilm, creating a feeding membrane. Feeding chambers were then inverted so that the aphids sat directly on top of their food source. Feeding chambers were enclosed inside an additional large petri dish with a layer of moistened filter paper to maintain a humid environment. A total of 14 aphids were used for each treatment. Adult mortality, nymph production and nymph survival were monitored for eight days. Single factor analysis of variance (ANOVA) was used to determine significance among the treatments. A 2-tailed t-test assuming unequal variance was used to determine significance between two groups.

### Results

The number of adults and nymphs surviving on perennial ryegrass-endophyte root-sucrose diet were determined over eight days. The symbiota tested were perennial ryegrass (prg)-SE (with standard endophyte), prg-AR37, prg-NEA2, 1137-RGT15 and 1137-RGT18. Prg-WE (without endophyte) was used as an endophyte-free control (Table 18).

TABLE 18
The mortality and fecundity of pasture root aphids exposed to a root-sucrose diet derived
from perennial ryegrass-endophyte symbiota. The total number and percentage of adults
surviving (A), nymphs born (NB) and nymphs surviving (NA) was assessed over eight days.

| | Day | 1 | | 2 | | 3 | | 4 | | 5 | | 6 | | 7 | | 8 | |
|---|---|---|---|---|---|---|---|---|---|---|---|---|---|---|---|---|---|
| Prg-WE¹ | A | 12 | 100% | 12 | 100% | 12 | 100% | 12 | 100% | 8 | 64% | 5 | 43% | 1 | 7% | 1 | 7% |
| | NB | 17 | | 30 | | 40 | | 48 | | 55 | | 55 | | 55 | | 55 | |
| | NA | 17 | 100% | 30 | 100% | 39 | 98% | 40 | 83% | 30 | 55% | 20 | 36% | 4 | 7% | 0 | 0% |
| Prg-SE | A | 14 | 100% | 14 | 100% | 11 | 79% | 8 | 57% | 6 | 43% | 4 | 29% | 3 | 21% | 0 | 0% |
| | NB | 12 | | 22 | | 28 | | 38 | | 39 | | 39 | | 39 | | 39 | |
| | NA | 12 | 100% | 22 | 100% | 18 | 64% | 20 | 53% | 21 | 54% | 11 | 28% | 1 | 3% | 0 | 0% |
| Prg- | A | 14 | 100% | 12 | 86% | 8 | 57% | 6 | 43% | 5 | 36% | 5 | 36% | 0 | 0% | 0 | 0% |
| AR37 | NB | 16 | | 28 | | 38 | | 44 | | 46 | | 46 | | 46 | | 46 | |
| | NA | 16 | 100% | 28 | 100% | 37 | 97% | 36 | 82% | 29 | 63% | 11 | 24% | 3 | 7% | 0 | 0% |
| Prg- | A | 13 | 93% | 12 | 86% | 11 | 79% | 10 | 71% | 7 | 50% | 4 | 29% | 1 | 7% | 0 | 0% |
| NEA2 | NB | 7 | | 14 | | 20 | | 25 | | 27 | | 27 | | 27 | | 27 | |
| | NA | 7 | 100% | 13 | 93% | 17 | 85% | 20 | 80% | 19 | 70% | 9 | 33% | 4 | 15% | 0 | 0% |
| 1137- | A | 13 | 93% | 11 | 79% | 11 | 79% | 5 | 36% | 4 | 29% | 3 | 21% | 2 | 14% | 0 | 0% |
| RGT15 | NB | 5 | | 13 | | 31 | | 32 | | 31 | | 31 | | 31 | | 31 | |
| | NA | 5 | 100% | 13 | 100% | 28 | 90% | 23 | 72% | 17 | 55% | 10 | 32% | 4 | 13% | 4 | 13% |
| 1137- | A | 13 | 93% | 13 | 93% | 13 | 93% | 5 | 36% | 5 | 36% | 5 | 36% | 3 | 21% | 1 | 7% |
| RGT18 | NB | 6 | | 20 | | 24 | | 28 | | 28 | | 28 | | 28 | | 28 | |
| | NA | 6 | 100% | 20 | 100% | 20 | 83% | 16 | 57% | 17 | 61% | 15 | 54% | 10 | 36% | 2 | 7% |

¹at the beginning of the experiment adult aphids were n = 12 for prg-WE, all other symbiota n = 14

Adult aphids exposed to prg-WE diets survived longer than those exposed to perennial ryegrass-endophyte symbiota (FIG. 12). For example, 37% of adult aphids exposed to 1137-RGT15 and 1137-RGT18 survived to four days, compared to 100% for prg-WE. Differences in adult survival at 4 days, for the symbiota tested, were statistically significant (ANOVA, P=0.0039). Significant differences (P<0.05) were observed between symbiota and prg-WE (adult aphid survival average and variance 1,0) at 4 days: prg-SE (0.571,0.264; P=0.008), prg-AR37 (0.438,0.264; P=0.0011), prg-NEA2 (0.714,0.220; P=0.040), 1137-RGT15 (0.357,0.247; P=0.0003) and 1137-RGT17 (0.357,0.247; P=0.0003). No significant differences (P<0.1) were determined between other pairwise combinations of prg-endophyte symbiota. All endophytes reduced adult survival. When considering ranking of symbiota for effectiveness in reducing adult survival 1137-RGT18=(1137-RGT15>prg-AR37)>prg-SE>prg-NEA2>prg-WE.

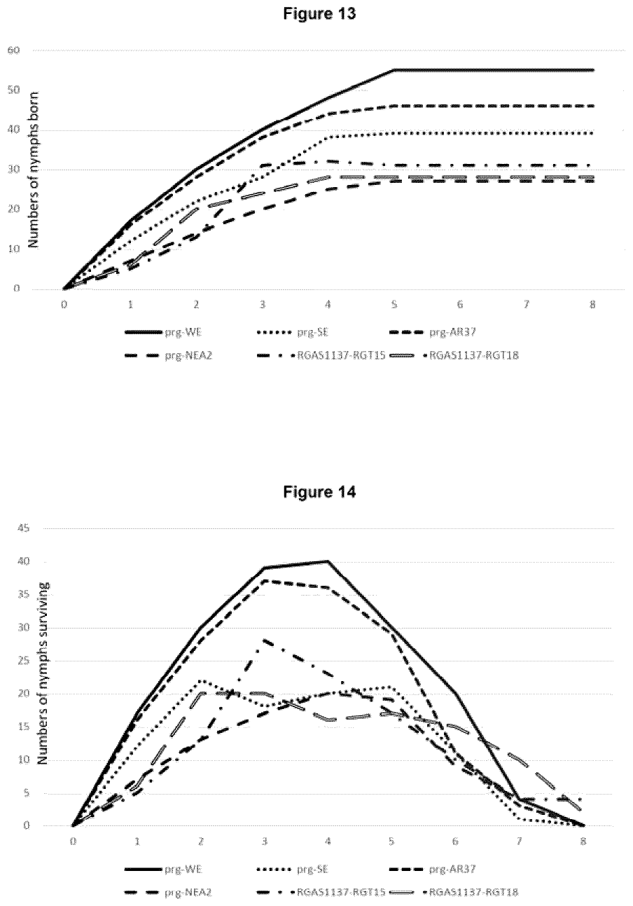

Aphid fecundity, measured as nymph production, ceased at five days (FIG. 13). Compared to prg-WE (average and variance for nymph production at 5 days 4.583,13.356), significantly (P<0.1) less nymphs were born when exposed to root-sucrose diet of perennial ryegrass-endophyte symbiota prg-NEA2 (1.929,5.918; P=0.045), 1137-RGT15 (2.214,5.72; P=0.071) and 1137-RGT18 (2,5.692; P=0.051) but not prg-AR37 (3.286,10.066; P=0.348) or prg-SE 2.786,7.412; P=0.176). No significant differences (P<0.1) were determined between other pairwise combinations of prg-endophyte symbiota. The symbiota 1137-RGT15, 1137-RGT18 and prg-NEA2 effectively reduced aphid fecundity. When considering ranking of symbiota for effectiveness in reducing fecundity (1137-RGT18=1137-RGT15=prg-NEA2)>(prg-SE=prg-AR37=prg-WE).

Maximum nymph survival was observed at four days (FIG. 14). Significant differences (P<0.1) were observed between symbiota (ANOVA, P=0.088). Compared to prg-WE (average and variance for nymph survival 3.33,8.42), significantly (P<0.1) less nymphs survived when exposed to prg-SE (1.429,2.72; P=0.06), prg-NEA2 (1.429,3.495; P=0.067), 1137-RGT15(1.643,3.324; P=0.098) and 1137-RGT18 (1.143,2.132; P=0.031) but not prg-AR37 (2.571,8.11; P=0.507). No significant differences (P<0.1) were determined between other pairwise combinations of prg-endophyte symbiota. The symbiota 1137-RGT15, 1137-RGT18, prg-SE and prg-NEA2 effectively reduced nymph survival. When considering ranking of symbiota for effectiveness in reducing nymph survival 1137-RGT18>(prg-SE=1137RGT15=prg-NEA2)>(prg-AR37=prg-WE).

### Conclusions

An in vitro bioassay was used to evaluate perennial ryegrass-endophyte symbiota for effectiveness in mealy grass root aphid (Aploneura lentisci) control. Aphids were fed a diet comprising a pooled sample of c. 100 symbiota root samples suspended in sucrose. Use of a pooled sample provides an indication of the symbiota average.

Significant differences in aphid mortality and fecundity were observed when aphids were fed the root-sucrose diet. The presence of an endophyte generally reduced aphid fitness compared to prg-WE. When considering ranking of symbiota for effectiveness in root aphid control RGAS1137-RGT18, RGAS1137-RGT15 and prg-NEA2 overall provided better whole-of-life cycle control than prg-SE and prg-AR37 see Table 18 and FIGS. 12-14.

### Example 9—Replicated Live Animal Feeding Trials Conducted Summer to Autumn using Lambs

### Materials and Methods

### Establishment and Pasture Management

The trial area was sprayed-out, double-disced and power harrowed prior to sowing. Drilling took place on April using a Horsch Drill, the 3 replicates of each treatment were drilled and then the drilled cleaned thoroughly before starting the next treatment. The paddocks, 0.17 hectares (36 m×48 m) in size, were sown as pure swards of perennial ryegrass.

The target sowing rate was 15 kg/ha of seed, assuming 90% germination and 90% field emergence and rates were adjusted according to seed size, in theory this would achieve a plant population of 520 plants/m².

Weeds were controlled through the winter and spring with applications of 650 mL/ha of Starane for winter application and a combination of Jaguar at 1.5 L/ha and Nortron at 4 L/ha for spring application.

The trial area received 250 kg/ha of Cropzeal 20 N as starter fertiliser before drilling in April 2018 and four applications of SustaiN; 100 kg/ha in September, 100 kg/ha at the end of October, 150 kg/ha at the end of December and 150 kg/ha at the end of January, totaling 500 kg/ha leading up to the trial period.

The trial was not irrigated. Grazing occurred in common by ewes for three weeks in November/December, with ewes removed on mid-December and again by lambs in January, with the lambs coming off on end of January and the trial area topped to 75 mm height after which point the blocks were fenced into individual paddocks.

### Climate

The summer of 2019 was hotter and dryer than the 10 year average. Mean air temperature and monthly rainfall from the Lincoln Broadfield EWS Weather Station showed that March of 2019 was on average 1.7° C warmer than the mean of 2009 to 2019.

TABLE 18.1
Mean air temperature and monthly rainfall from the Lincoln
Broadfield EWS Weather Satation for the summer of 2019

| | JANUARY | | FEBRUARY | | MARCH | |
|---|---|---|---|---|---|---|
| | | 10 Year | | 10 Year | | 10 Year |
| | 2019 | Mean | 2019 | Mean | 2019 | Mean |
| Total | 36.2 | 43.7 | 29.2 | 41.7 | 26.8 | 47.5 |
| Rainfall (mm) | | | | | | |
| Mean Air | 18.6 | 17.3 | 17.8 | 17.0 | 17 | 15.3 |
| Temperature | | | | | | |

Endophyte Presence 50 tiller samples were taken from each paddock and endophyte levels were measured and before further testing were confirmed to be above the 70% threshold for selling commercial seed to the market refer the KASP section and Table 19 below.

### KASP

Endophyte purity and content testing was performed (44-50 tillers from each sample) from the perennial ryegrass-endophyte lines (3 replicates) were tested.

DNA was extracted from each line using the MagAttract Plant DNA kit (Qiagen) in an automated workflow with liquid handling platforms (Beckman Coulter). Each DNA sample was genotyped using a strain specific ‘Kompetitive Allele Specific PCR’ (KASP™) genotyping method using diagnostic SNPs. Each plate contained eight positive controls as well as four no template controls (without DNA) for quality control. Each sample was tested for presence of the expected endophyte.

TABLE 19
Mean Endophyte Presence (%) of treatments

| Cultivar Endophyte | |
|---|---|
| Combination | KASP |
| SAMSON AR1 | 98 |
| STELLAR-RGT18 | 95 |
| SAMSON SE | 99 |
| TROJAN NEA2 | 83 |
| SAMSON AR37 | 75 |
| STELLAR-RGT15 | 73 |

Note:
KASP test provides the most definitive test for level of presence of live endophyte.
Commercial levels for live endophyte are a minimum of 70%

The key controls used for the trials were SAMSON AR1 (Negative control, AR1, nil animal health effects) and SAMSON SE (Positive standard endophyte control).

Yield for the cultivars detailed in Table 19 was assessed as a function of calculating the number of lambs to be placed into each paddock at the start of the trial. The yield was measured at the mid-point of the trial to ensure enough feed would be available to lambs for the remainder of the trial period-The close range of the final yield measurements (890 to 1134 kg DM/ha) shows that lamb numbers per paddock were well managed and that the assumptions made in the calculations for these were sound.

### Lamb Liveweight Gain

SAMSON SE had the lowest liveweight gain in weeks 0-2 and varieties 2, 3, 5, 6 and 8 had significantly higher gains than this. There was only one significant difference in weeks 2-4 for liveweight gain.

TABLE 20
Mean lamb liveweight change (kg/head)
for Week 0-2, Week 2-4 and Week 0-4.

| | Mean lamb liveweight change (kg/head) | | | | | |
|---|---|---|---|---|---|---|
| | Week 0- | | Week 2- | | Week 0- | |
| Treatment | Week 2 | | Week 4 | | Week 4 | |
| STELLAR-RGT18 | 6.1 | a | 0.6 | ab | 6.7 | a |
| STELLAR-RGT15 | 5.2 | ab | 1.1 | a | 6.3 | a |
| SAMSON AR37 | 6.3 | a | −0.3 | ab | 6 | ab |
| SAMSON SE | 4.4 | b | 0.1 | ab | 5.9 | ab |
| SAMSON AR1 | 5.4 | ab | −0.3 | ab | 5.1 | ab |
| TROJAN NEA2 | 5.3 | ab | −0.9 | b | 4.4 | b |

| | | | |
|---|---|---|---|
| LSD 5% | 1 | 1 | 1 |

Liveweight gain as a measure of the potential treatment effect is not a useful gauge as it is dependent on the number of lambs per paddock which, in this case, was according to the ETC Protocol minimum lambs/paddock requirement rather than a calculated number of lambs based on dry matter production available.

### Stock and Animal Ethics

Animal Ethics Approval was sought and granted by the Lincoln University Animal Ethics Approval Committee.

Trial lambs were sourced from the neighbouring farmer who made available 320 Coopworth x Hampshire cross and straight Coopworth ewe lambs at an average liveweight (unfasted) of 31.3 kg/head. All lambs had been grazed on Lucerne for the three weeks prior to the start of the trial, were freshly shorn the previous week and received a drench prior to going onto the trial. Lambs were allocated to paddocks at random and were weighed prior to going onto the trial.

### Feed Grown

Yield was assessed by taking 3 strips using a rotary mower to 4 cm from each paddock and drying a sub sample to calculate dry matter percent.

Several assumptions were made when using yield figures to calculate the number of lambs to be allocated to each paddock: that the predicted dry matter production growth per day during the trial period would be 35 kg DM/ha, that the residual remaining at the end of the trial period would be 800 kg DM/ha and that lamb intake during the trial period would be 1.5 kg DM/ha/head.

Using these calculations, it was expected that feed would be tight towards the end of the 28 day trial period but 10 lambs per paddock (the ETC Protocol minimum number of lambs/paddock) were applied to all paddocks except three paddocks which had higher yields, these received 12 lambs.

Yield measurements were also taken using the same method, however the final measurement, at the end of the trial period was taken using a rising plate meter as a rotary mower would not have collected much yield above the 4 cm cutting height.

Yield was closely monitored and at the mid-point of the trial, those paddocks that had 12 lambs allocated to them had 2 lambs removed as the pasture cover was getting low. The mid-point yield measurement highlighted four other paddocks with low pasture covers (<1000 kg DM/ha). The Biometrician David Baird was consulted and his recommendation was to remove some lambs from each of these paddocks (as opposed to running with all 10 lambs and shortening the trial period for those paddocks), so three lambs were removed to leave seven in each paddock.

### Lamb Liveweights

Unfasted lamb liveweights were recorded at the start, mid-point and end of the trial period respectively.

Herbage Alkaloids—See also Example 10 Below

Samples for herbage alkaloid analysis were taken using an electric handpiece at the start, mid-point and end of the trial period respectively). Ten areas approximately 20 cm long were cut to ground level and combined from which a sub-sample of 150 grams was collected and stored immediately within layers of ice. Samples were then transferred to a freezer.

Samples were freeze dried and then referenced under trial codes and forwarded for laboratory analysis.

### Staggers Scoring

Lambs were assessed using the Keogh Scale (see below) after grazing on paddocks for one week, again after two weeks after which point the scoring occurred twice weekly.

While operating in the best interests of the trial animals, the Trial Operator removed any lambs from the trial that staggered to the point of falling over (those that registered as a Score 4 or 5 on the Keogh Scale).

The Trial Operator interpreted a Score 3 to include ‘stiff leg walking’ or ‘knee-walking’ and a Score 4 ‘knee-walking to the point of falling over’.

### Keogh (1973 Description of Staggers Symptoms)

0—No symptoms
1—Slight trembling of neck, shoulders, and flank muscles after hard exercise (400 m run).
2—Marked trembling of neck, shoulders and flank muscles, and shaking of head after hard exercise, but no lack of co-ordination.
3—Marked trembling of general musculature and head shaking; some lack of co-ordination of movement and impaired vision while running.
4—Muscle tremors and head shaking after a short run (<30 m) or sudden disturbance; continued exercise elicits a marked lack of co-ordination resulting in a characteristic staggering gait which normally ends with the animal falling down; a short period of moderate to severe muscular spasm follows, after which the animal is able to regain its feet and walk off.
5—Severe muscle tremors elicited by slight disturbance or exercise <<10 m rapid movement) which invariably result in staggering and collapse in a severe tetanic spasm which may last up to 20 min in very bad cases.

### Staggers Results

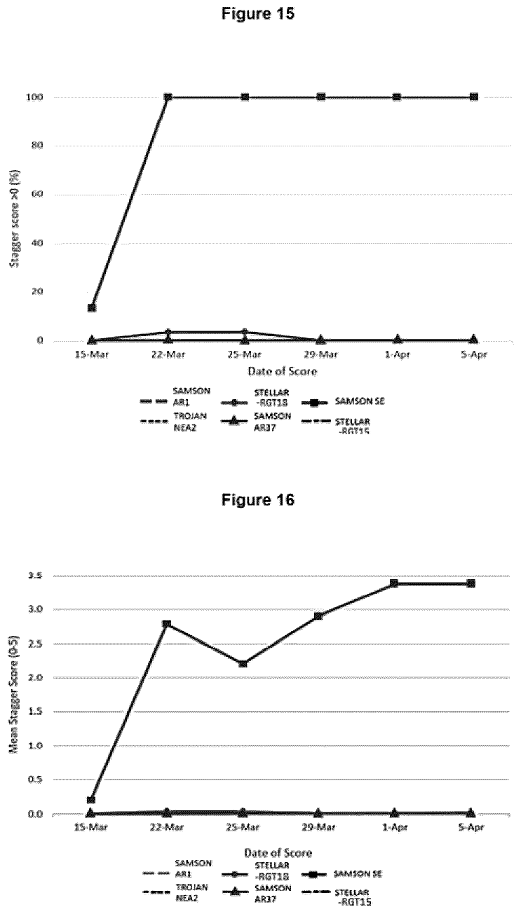

Referring to Table 20 above and FIGS. 15 and 16 what is significant is that the only treatment that showed a significant increase in stagger scores was SAMSON SE.

In particular SAMSON SE was significantly higher on the Keogh Scale than all other treatments at the 5% level at the first score (measured 7 days after the start of the trial) and was significantly higher again at the 0.1% level when next measured 10 days later.

Indeed, the observed staggers in SAMSON SE paddocks was so extreme Score 3 and above that all lambs were removed from the paddocks prior to the conclusion of the trial.

There were no other significant differences between the remaining 5 treatments as can be seen from the overlapping baseline of their datapoints in Table 20 and FIGS. 15 and 16—(with only a slight variance clearly observable for STELLAR-RGT18 in FIG. 16). Importantly, where lambs have been removed from plots the previous stagger scores have been carried over, to allow unbiased estimates of effects.

### Conclusion

It is clear from the above results that RGT15 and RGT 18 endophytes of the present invention present a real-world advantage to SAMSON SE wildtype endophyte symbiont in terms of not sacrificing animal health for any improved reduction in insect related predation over non-symbiont grass.

### Example 10—Alkaloid Results

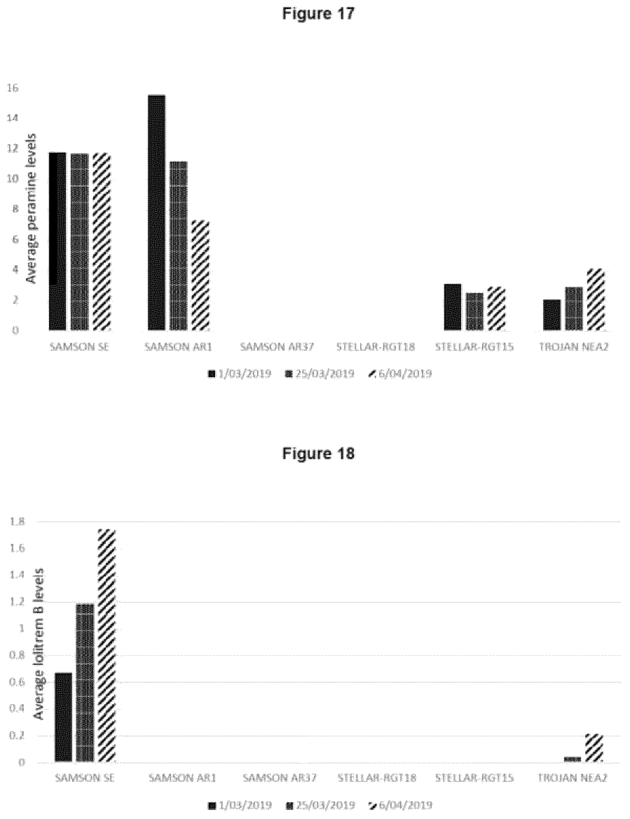

The results of the herbage alkaloids from the endophyte grass symbionts (treatments) tested as mentioned above are shown in FIGS. 17-19.

As can be seen in FIG. 17 STELLLAR-RGT15 produces a similar amount of peramine to TROJAN NEA2 with much higher levels being observed in SAMSON AR1 and SAMSON SE.

In FIG. 18 it can be seen that only SAMSON SE produces significant levels of lolitrem B compared to the relatively small amounts produced by TROJAN NEA2 with no other treatments producing any detectable levels or lolitrem B.

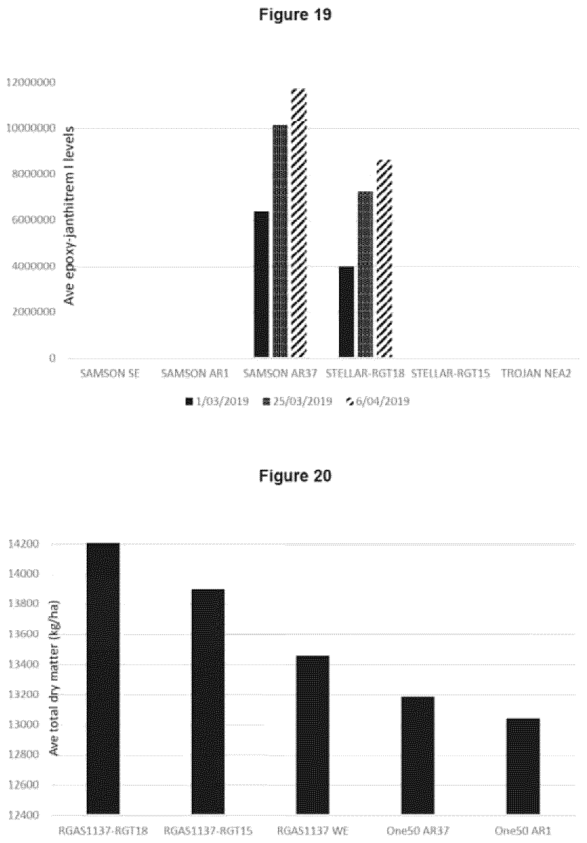

In FIG. 19 the SAMSON AR37 and STELLAR-RGT18 treatments are the only grass endophyte symbionts producing epoxy-janthitrem I. With STELLAR-RGT18 producing around two thirds the amount of epoxy-janthitrem I of that produced by SAMSON AR37.

It is noteworthy, as can be seen from FIGS. 4 and 5 that the RGT18 grass symbiont in addition to epoxy-janthitrem I, also produces epoxy-janthitrems II, III and IV at relative ratios, that are such that:
the combination of epoxy-janthitrems I-IV produced by the RGT18 grass symbiont does not impact animal performance; yet
clearly imparts black beetle resistance to the grass;
refer Examples 7 and 9 above.

### Conclusion

The only grass endophyte symbiont tested which produced significant amounts of lolitrem B was SAMSON SE, importantly neither STELLAR-RGT15 nor STELLA-RGT18 produced lolitrem B.

It can be seen from the results that STELLAR-RGT15 is an endophyte capable of producing commercial acceptable levels of peramine.

The results also show that both STELLAR-RGT18 and SAMSON AR37 are the only grass endophyte symbionts which produce epoxy-janthitrem I.

### Example 11 Agronomy: Grass Endophyte Symbiont Dry Matter Production Studies

### Materials and Methods

The Lennox Trial compared dry matter production of RGA1137-RGT18, RGA1137-RGT15, RGA1137 WE, One50 AR1, and One50 AR37. The trial location was situated near Ladbrooks, Canterbury with Wakanui clay loam soil. It was a dryland and mow only trial. The randomized complete block having 4 replicates with nutrients being replaced following each harvest based on the mean yield removal. The Waikeria Trial compared dry matter production of STELLAR-RGT18, STELLAR-RGT15, STELLAR AR1, Request AR1, and Request AR37. The trial location was situated in the Waikato region near Te Awamutu with ash (allophanic) soil type. It was a dryland and mow only trial. The randomized complete block having 4 replicates with nutrients being replaced following each harvest based on the mean yield removal. Both trials were harvested when the average dry matter yield was between 2000-2700 kg/ha. Fresh weight was recorded, with a subsample of the fresh herbage dried to provide a dry matter %.

### Results

Results of the Lennox Trial are shown in Table 21 and FIG. 20, and these clearly indicate that average total dry matter yields of host plants containing RGT15 or RGT18 were superior over those plants with no endophytes, or with the commercial endophytes tested.

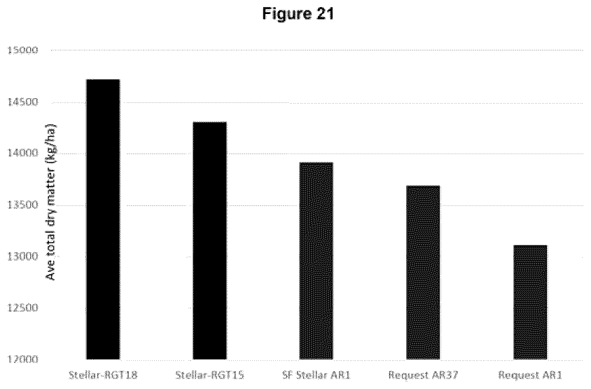

Results of the Waikeria Trial are shown in Table 22 and FIG. 21, and these also clearly indicate that average total dry matter yields of host plants with RGT15 or RGT18 were superior over those plants with no endophytes, or containing the commercial endophytes tested.

TABLE 21
Lennox Trial: Average Total Dry
Matter Yield (kgDM/ha) by Years

| | | | | Average Total |
|---|---|---|---|---|
| | Year | Year | Year | Dry Matter |
| Variety | 1 | 2 | 3 | Yrs 1-3 |
| RGAS1137-RGT18 | 15,450 | 12,321 | 14,426 | 14,203 |
| RGAS1137-RGT15 | 14,946 | 12,778 | 13,767 | 13,897 |
| RGAS1137 WE | 14,279 | 11,566 | 14,219 | 13,456 |
| One50 AR37 | 14,357 | 12,470 | 12,763 | 13,183 |
| One50 AR1 | 14,423 | 11,705 | 12,971 | 13,043 |
| Trial Mean | 13,850 | 11,830 | 13,399 | 13,009 |
| LSD (5%) | 1,206 | 1,072.0 | 1,050.0 | 611.9 |
| CV % | 9.3 | 5.4 | 6.6 | 5.1 |

TABLE 22
Waikeria Trial: Average Total Dry
Matter Yield (kgDM/ha) by Years

| | | | Average Total |
|---|---|---|---|
| | | | Dry Matter |
| Variety | Year 1 | Year 2 | Yrs 1-2 |
| STELLAR-RGT18 | 12,212 | 17,103 | 14,717 |
| STELLAR-RGT15 | 11,702 | 16,797 | 14,297 |
| SF STELLAR AR1 | 11,370 | 16,221 | 13,910 |
| REQUEST AR37 | 11,215 | 16,006 | 13,683 |
| REQUEST AR1 | 10,656 | 15,432 | 13,111 |
| Trial Mean | 11,617 | 16,282 | 13,963 |
| LSD (5%) | 577.4 | 773.6 | 761.7 |
| CV % | 6.7 | 3.3 | 4.3 |

### Conclusion

The results of both the Lennox and Waikeria trials show RGT15 and RGT18 endophytes support better yields over a three year period within the same grass cultivar.

The invention detailed herein may provide one or more advantages over the prior art endophyte grass symbionts and/or wildtype grasses, or at least offer the public a useful choice.

The invention as detailed above may also be said broadly to consist in the parts, elements and features referred to or indicated in the specification of the application, individually or collectively, in any or all combinations of two or more of said parts, elements or features.

Aspects of the present invention have been described by way of example only and it should be appreciated that modifications and additions may be made thereto without departing from the scope thereof as defined in the appended claims.

### REFERENCES

[1] van Zijll de Jong E., Dobrowolski M.P., Bannan N.R., Stewart A.V., Smith K.F., Spangenberg G.C., and Forster J.W., 2008. Global Genetic Diversity of the Perennial Ryegrass Fungal Endophyte Neotyphodium lolii. Crop Science, 48:1487-1501.
[2] Gallagher R.T., White E.P., and Mortimer P.H., 1981. Ryegrass staggers: Isolation of potent neurotoxins lolitrem A and lolitrem B from stagger-producing pastures. N.Z. vet. J. 29:189-190.
[3] Yates S.G., Plattner R.D., and Garner G.B., 1985. Detection of ergopeptine alkaloids in endophyte infected, toxic Ky-31 tall fesue by mass spectrometry/mass spectrometry. J. Agric. Food Chem. 33:719-722.
[4] Ball O.J.P., Miles C.O., and Prestidge R.A., 1997. Ergopeptine alkaloids and Neotyphodium lolii-mediated resistance in perennial ryegrass against adult Heteronychus arator (Coleoptera, Scarabaeidae). J. Econ. Entomol. 90:1382-1391.
[5] Prestidge R.A. and Gallegher R.T., 1988. Endophyte fungus confers resistance to ryegrass: Argentine stem weevil larval studies. Ecol. Entomol. 13:429-435.
[6] Rowan D.D. and Gaynor D.L., 1986. Isolation of feeding deterrents against Argentine stem weevil from ryegrass infected with endophyte Acremonium loliae. J. Chem. Ecol. 12:647-658.
[7] Morland Latch G.C., Christensen M.J., Tapper B.A., Easton H.S., Hume D.E., Fletcher L.R. 2000. Ryegrass endophytes. U.S. Pat. No. 6,072,107.
[8] Cameron N.E., 2015. Grass endophyte. U.S. Pat. No. 9,133,434.
[9] Tapper B.A., Cooper B.M., Easton H.S., Fletcher L.R., Hume D.E., Lane G.A., Morland Latch G.C., Lee Pennell C.G., Popay A.J., Christensen M.J., 2011. Grass endophytes. U.S. Pat. No. 7,976,857.
[10] Finch S. C., Wilkins A. L., Popay A. J., Babu J. V., Tapper B. A., Lane G. A. (2010). “The isolation and bioactivity of epoxy-janthitrems from AR37 endophyte-infected perennial ryegrass,” in Proceedings of the 7th International Symposium on Fungal Endophytes of Grasses, eds Young C. A., Aiken G., McCulley R. L., Strickland J., Schardl C. L., editors. (Lexington, KY).
[11] Hennessy L. M. (2015). Epoxy-Janthitrems, Effects of Temperature on in Planta Expression and their Bioactivity against Porina Larvae. Masters thesis, University of Waikato, Hamilton.
[12] Latchs G., and Christensen M. 1985. Artificial infection of grasses with endophytes. Annals of Applied Biology: An International Journal of the AAB., 107(1), 17-24.
[13] Keogh, R.G. 1973. Induction and prevention of ryegrass staggers in grazing sheep. New Zealand Journal of Experimental Agriculture 1:55-57.

What is claimed is:

1. A method of improving the viability, persistence, and/or vigor of a host grass, the method comprising:
artificially inoculating the host grass with an RGT15 (deposit accession number V18/011210) to produce at least one alkaloid or combination of alkaloids that provide the host plant with a favorable alkaloid profile and/or protect the plant from biotic stresses;
whereby the LpTG-1 endophyte RGT15 strain produces the at least one alkaloid or combination of alkaloids that provide the host plant with the favorable alkaloid profile and/or protect the host plant from the biotic stresses,
wherein the host grass is selected from the group consisting of Lolium multiflorum, Lolium x hybridum, and Lolium perenne.

2. The method of claim 1, wherein the alkaloid is at least one janthitrem epoxide compound.

3. The method of claim 2, wherein the alkaloid is selected from the group consisting of epoxy janthitrems I-IV and combination thereof.

4. The method of claim 3, wherein the production of any one of epoxy janthitrems I-IV is greater than that of an LpTG-1 endophyte AR37 strain in summer.

5. A production zone which includes growing therein at least one host grass, wherein the host grass is selected from the group consisting of Lolium perenne, Lolium multiflorum, and Lolium x hybridum, wherein the host grasses have had their viability, persistence and/or vigor improved by the method of claim 1.

6. The production zone of claim 5, wherein two of the at least one host grass are randomly interspersed within the production zone.

7. The production zone of claim 5, wherein two of the at least one host grass are grown in discrete regions within the production zone.

8. The production zone of claim 7, wherein the two of the at least one host grass are each grown in a respective half of the production zone.

9. The production zone of claim 5, wherein an average total dry matter yield from the production zone, over a period of one to three years, is greater than that of a production zone with host grass(es) inoculated with an LpTG-1 endophyte AR37 strain.

10. An isolated endophyte of Epichloë festucae var. lolii species LpTG-1 RGT1lptg5 deposited at National Measurement Institute and accorded accession number V18/011210.
